(19) United States
(12) Patent Application Publication
(10) Pub. No.: US 2002/0048566 A1
(43) Pub. Date: Apr. 25, 2002

(54) Modulation of cellular apoptosis and methods for treating cancer

(76) Inventors: Wafik S. El-Deiry, Bryn Mawr, PA (US); Eric J. Bernhard, Philadelphia, PA (US); Timothy F. Burns, Philadelphia, PA (US); E. Robert McDonald III, Philadelphia, PA (US)

Correspondence Address: NATH & ASSOCIATES, 1030 15th STREET, 6TH FLOOR, WASHINGTON, DC, 20005, US

(21) Appl. No.: 09/950,836

(22) Filed: Sep. 13, 2001

Related U.S. Application Data
(60) Provisional application No. 60/232,556, filed on Sep. 14, 2000.

(51) Int. Cl. A61K 48/00; A61K 38/17; C12N 15/86

(52) U.S. Cl. 424/93.21; 435/320.1; 514/12

(57) ABSTRACT

This invention relates to modulating cellular apoptosis in an animal by regulating the quantity of functional KILLER/DR5 receptor protein molecules in the target cell(s) of said animal, increasing the effectiveness of certain therapies such as chemotherapy and radiotherapy. Such modulation increases the difference in toxicity response between target cell(s) and non-target cell(s) in response to a therapy or treatment and comprises upregulation of wild-type proteins in cells targeted for increased apoptosis or upregulation of loss-of-function proteins in cells targeted for decreased apoptosis. The methods of this invention comprise administering a nucleic acid sequence encoding a wild-type KILLER/DR5 receptor protein or a nucleic acid sequence encoding a loss-of-function mutant KILLER/DR5 receptor protein to said animal. The pharmaceutical compositions of this invention comprise a loss-of-function KILLER/DR5 receptor protein. The isolated nucleic acid sequences of this invention comprise wild-type KILLER/DR5 receptor nucleic acid sequence(s) bearing one or more point mutations at selected amino acid(s). A target of this invention is spleen and gastrointestinal cancer cells.

This application claims the benefit of U.S. Provisional Patent Application No. 60/232,556, filed Sep. 14, 2000, the contents of which is hereby incorporated by reference.

This work was supported in part by National Institutes of Health Grants CA 75454 (W S El -Deiry) and CA 75138 (EJ Bernhard and W S El-Deiry). The United States government may have rights in this invention by virtue of this support.

### BACKGROUND OF THE INVENTION

1. Field of Invention

The present invention relates to novel methods for modulating cellular apoptosis by increasing the difference in toxicity response between a target cell and a non-target cell. In particular, the present invention relates to methods for inducing apoptosis in cancer cells and/or decreasing apoptosis in non-cancer cells, in order to increase the effectiveness and decrease the adverse side-effects of cancer treatments such as radiation therapy or chemotherapy. The method is exemplified by the use of wild-type KILLER/DR5 receptor protein to induce apoptosis in human liver, spleen, and gastrointestinal tumor cells.

2. Background

In the effort to develop cancer novel therapies, one promising approach is to identify targets that are different between normal cells and cancer cells. The present invention arises from the tissue specificity of cell death responses, a feature that may be exploited in strategies to widen the therapeutic window of combination cancer therapies. In p53-targeted cancer therapy, novel strategies to enhance or block specific effectors are designed to improve therapeutic outcome.

Apoptosis is an active and programmed process for eliminating superfluous, altered, or malignant cells, which can be initiated by a variety of endogenous and exogenous environmental stimuli. One of the striking features of apoptosis is phagocytosis of apoptotic cells by their neighbors. Therefore, apoptosis causes much less destruction of tissue than necrosis, the non-physiological type of cell death. There is evidence that inhibition of the apoptotic processes can be important in tumor formation. As a result, the elimination of tumor cells via induction of apoptotic cell death has become a promising approach in experimental cancer therapy. A variety of chemotherapeutic compounds and ionizing radiation have been demonstrated to induce apoptosis in tumor cells, in many instances via wild-type p53. Apoptosis is characterized by shrinkage of cells, segmentation of the nucleus, condensation and eventual internucleosomal degradation of DNA. An apoptotic pathway can be characterized by its mediator(s) and represser(s). It has been shown that the tumor suppressor protein p53 can act as a positive regulator of apoptosis. Expression or overexpression of wild-type p53 can induce apoptosis in a number of cell types.

Conventional therapies such as radiation treatment and chemotherapy rely on the apoptotic physiological response to the cellular damage caused by radiation or toxic chemicals. Apoptosis-inducing pathway(s) which has been the target of such therapies are dependent upon the p53 protein. In addition, subject to particular inducing agents or cellular backgrounds, the induction of apoptosis can also take place in a p53-independent manner. Unfortunately, a large number of tumors acquire a mutation in p53 during their development and become resistant to p53-dependent therapy. It therefore is of interest to identify compositions and methods by which to induce both p53-dependent and p53-independent apoptosis as a means of inhibiting tumor cell growth.

p53, Cancer, and Therapy

The p53 gene is the most commonly altered gene in human cancer, being involved through mutation in over 50% of all of human cancers world-wide. It is now believed that the vast majority of the cancers without mutational inactivation of p53 have alterations in the p53 pathway (Vogelstein B, et al., Surfing the p53 network, Nature 408:307-310 (2000) and Schmitt C A, et al., Genetic analysis of chemoresistance in primary murine lymphomas, Nature Med. 6: 1029-1035 (2000)). Because p53 is the also the most commonly mutated gene in human cancer and the p53 pathway is involved in the vast majority of tumors without mutations in p53, this p53 protein becomes an ideal target for therapeutic development in cancer.

p53 abnormalities represent a fundamental difference between cancer cells and normal cells and as such they can be exploited in therapeutic design. Besides being an ideal target, p53 and its ability to induce tumor growth suppression through cell cycle arrest and the induction of apoptosis has been implicated in the cellular response to DNA-damaging radiotherapy and chemotherapy (Vogelstein B, et al., supra, and Schmitt C A, et al., supra). The emerging understanding of the pathways upstream and downstream of p53, and their signal- or tissue-specificity is providing a basis to develop more effective therapies. In addition to targeting the p53 pathway, there are emerging strategies that target the p53 molecule itself. These include small molecules that can inhibit wild-type p53 function or other agents that are capable of restoring wild-type p53 function in human cancer cells with mutant p53 protein (Komarov P G, et al., A chemical inhibitor of p53 that protects mice from the side effects of cancer therapy, Science 285:1733-1737 (1999) and Foster B A, et al., Pharmacological rescue of mutant p53 conformation and function, Science 286:2507-2510 (1999)). Yet other strategies are targeting key protein-protein interactions of p53 that disrupt its function in cancer (Kussie P H, et al., Structure of the MDM2 oncoprotein bound to the p53 tumor suppressor transactivation domain, Science 274:948-953 (1996); Stoll R, et al., Chalcone derivatives antagonize interactions between the human oncoprotein MDM2 and p53, Biochemistry 40:336-344 (2001); and Beerheide W, et al., Inactivation of the human papillomavirus-16 E6 oncoprotein by organic disulfides, Bioorganic Medic. Chem 8:2549-2560 (2000)).

The cytokine Tumor necrosis factor-related Apoptosis Inducing Ligand (“TRAIL”) and its signaling pathway is of interest for cancer therapy for a number of reasons. There are few agents that are truly cancer cell specific in terms of efficacy for cell death induction. TRAIL is an example of a molecule which specifically kills transformed and cancer cells but not most normal cells (Ashkenazi A, et al., Death receptors: Signaling and modulation, Science 281:1305-1308 (1998)). This property has stimulated much work to understand the difference between the effect of TRAIL towards normal versus cancer cells, as well as efforts to understand defects in TRAIL signaling in cancer cells that fail to respond to its cytotoxic effects. TRAIL has been well characterized; both isolated full length and fragment DNA sequences, and methods for transforming cultured cells for the production of the TRAIL polypeptide are the subject of U.S. Pat. No. 5,763,223 to Wiley, et al.

Applicants have found that one of the TRAIL receptors, KILLER/DR5, is implicated in the cellular response to DNA damaging radiation or chemotherapy as a target of p53 (Wu G S, et al., KILLER/DR5, a novel DNA-damage inducible death receptor gene, links the p53-tumor suppressor to caspase activation and apoptotic death, Adv. Exp. Med. Biol. 465:143-151 (2000)). Thus, although one of the attractive features of TRAIL is its ability to kill cancer cells with mutations in the p53 gene, the combination of TRAIL with chemotherapeutic agents has been found to be particularly effective in killing cancer cells with wild-type p53, presumably through induction of KILLER/DR5 expression (Kim K H, et al., Molecular determinants of response to TRAIL in killing of normal and cancer cells, Clin. Cancer Res. 6:335-346 (2000) and Kim K, et al., Enhanced TRAIL sensitivity by p53 overexpression in human cancer but not normal cell lines, Int. J. Oncol. 18:241-247 (2001)).

p53 is a potent tumor suppressor gene such that if added back into cancer cells it suppresses growth despite other molecular genetic changes in the cells (Baker S J, et al., Suppression of human colorectal carcinoma cell growth by wild-type p53, Science 249:912-915 (1990) and Blagosklonny M V, et al., Acute overexpression of wt p53 facilitates anticancer drug-induced death of cancer and normal cells, Int. J. Cancer 75:933-940 (1998)). This is particularly relevant and important as a therapeutic strategy, for example in the gene therapy of cancer. p53 induces growth arrest and/or apoptosis either when exogenously administered or through its involvement in the cellular response to DNA damage.

In addition to clearly playing a role in cancer susceptibility, p53 also appears to be a major determinant of sensitivity of cells to chemo- and radiotherapy (Lowe S W, et al., p53 status and the efficacy of cancer therapy in-vivo, Science 266: 807-810 (1994)).

Regulation, Structure, and Signals Downstream of p53

Clear and distinct signaling pathways are emerging upstream of p53 stabilization in response to a number of stresses (Ryan K M, et al., Regulation and function of the p53 tumor suppressor protein, Curr. Opin. Cell Biol. 13:332-337 (2001) and Sherr C J, et al., The ARF/p53 pathway, Curr. Opin. Genet. Dev. 10:94-99 (2000)). There appear to be at least three or more main pathways upstream of p53 (Vogelstein B, et al., supra, and El-Deiry W S, Regulation of p53 downstream genes, Semin. Cancer Biol. 8:345-357 (1998)).

First, the now classical DNA damage response pathway involves kinases that become “activated” through unclear mechanisms upon cellular exposure to DNA damaging agents such as ionizing radiation or topoisomerase II inhibitors (FIG. 2). These DNA damaging agents cause breaks in the cellular DNA which are believed to be an initiating signal. Once “activated” the kinases ATM or CHK2 can each phosphorylate p53 on N-terminal residues within its transactivation domain, thereby leading to increased p53 activity. Specifically, physical association between p53 and ATM leads to phosphorylation of p53, which is now believed to enhance its transcriptional activity rather than leading to an increase in its half-life per se (Canman C E, et al., Activation of the ATM kinase by ionizing radiation and phosphorylation of p53, Science 281:1677-1679 (1998)). ATM also acts on CHK2 to phosphorylate it (Kastan M B, et al., The many substrates and functions of ATM, Nature Rev. Mol. Cell. Biol. 1:179-186 (2000)). CHK2 in turn phosphorylates p53, which disrupts its binding to its negative regulator, E3 ubiquitin ligase MDM2. Release of MDM2 from p53 leads to p53 protein stabilization, which in turn allows p53 to mediate its potent downstream effect on cell growth suppression (El-Deiry W S, Regulation of p53 downstream genes, Semin. Cancer Biol. 8:345-357 (1998)).

DNA damage initiated signals leading to activation of ATM and CHK2 have recently been recognized to also target the phosphorylation of MDM2, further inhibiting MDM2:p53 interaction (Khosravi R, et al., Rapid ATM-dependent phosphorylation of MDM2 precedes p53 accumulation in response to DNA damage, Proc. Natl. Acad. Sci. USA 96:14973-14977 (1999)). In addition to targeting p53 for ubiquitin-mediated proteolysis, and directly interacting with and physically blocking the transactivation domain of p53, MDM2 interaction also targets p53 for nuclear export, thereby also facilitating its degradation and inhibiting its effects on gene expression in the nucleus (Geyer R K, et al., The MDM2 RING-finger domain is required to promote p53 nuclear export, Nature Cell Biol. 2:569-573 (2000)). It is of interest that the degradation of p53 by MDM2 utilizes different structural requirements within p53 as compared to human papillomavirus E6-targeted degradation (Hengstermann A, et al., Complete switch from Mdm2 to human papillomavirus E6-mediated degradation of p53 in cervical cancer cells, Proc. Natl. Acad. Sci. USA 98:1218-1223 (2001)).

A second p53 pathway, involving the ARF protein, appears to constitute the cellular response to inappropriate growth signals, the so-called “oncogene checkpoint.” For over two decades, it has been observed that p53 protein levels are high in transformed cells. For example, p53 was originally isolated as a highly expressed protein that bound to SV40 large T-antigen in transformed cells or as an aberrantly expressed “tumor antigen.” It was also known that a number of cellular and other viral oncogenes can increase the expression level of cellular p53. These include c-Myc, ras, and adenovirus E1A proteins. It has recently become clear that ARF, the alternative-reading frame at the INK4A locus, is a protein that interacts with MDM2, thereby sequestering it in nucleolar structures away from p53, which can then become more stable (Sherr C J, et al., The ARF/p53 pathway, Curr. Opin. Genet. Dev. 10:94-99 (2000)). There is recent evidence that ARF has p53-independent, MDM2-dependent effects on growth suppression through an as yet unclear mechanism (Weber J D, et al., p53-independent functions of the p19(ARF) tumor suppressor, Genes Dev. 14:2358-2365 (2000)). There is also evidence for MDM2-independent nuclear export of p53 following DNA damage through a newly discovered nuclear export signal in the amino-terminal region of p53 (Zhang Y, et al., A p53 amino-terminal nuclear export signal inhibited by DNA damage-induced phosphorylation, Science 292:1910-1915 (2001)).

A third p53 pathway is triggered by exposure to UV radiation. This UV-initiated p53 stabilization does not require ATM, but is believed to utilize a related kinase called ATR (Shiloh Y, ATM and ATR: networking cellular responses to DNA damage, Curr. Opin. Genet. Devel. 11:71-77 (2001) and Brown E J, et al., ATR disruption leads to chromosomal fragmentation and early embryonic lethality, Genes Dev. 14:397-402 (2000)). DNA replication blockade also triggers p53 stabilization in an ATM-independent manner (Gottrfredi V, et al., p53 accumulates but is functionally impaired when DNA synthesis is blocked, Proc. Natl. Acad. Sci. USA 98:1036-1041 (2001) and Takimoto R, et al., DNA replication blockade impairs p53-transactivation, Proc. Natl. Acad. Sci. USA 98:781-783 (2001)). The ultimate effect of the signals upstream of p53 appear to be lead to release of MDM2 from p53. This is the basis of a drug development strategy whose aim is to deliver peptides or small molecules into cells that still have wild-type p53 but which is believed to over express MDM2. Such peptides which fit into the p53:MDM2 interacting groove are expected to displace MDM2 thereby stabilizing the tumor suppressor p53 (Chene P, et al., A small synthetic peptide, which inhibits the p53-hdm2 interaction, stimulates the p53 pathway in tumour cell lines, J. Mol. Biol. 299:245-253 (2000)).

53 Pathways of Apoptosis and Cancer Therapy

The transcriptional targets upregulated by p53 which an in turn induce apoptosis fall into at least three categories (Vogelstein B, et al., supra and El-Deiry W S, supra):

(1) death domain containing proteins, including two proapoptotic death receptors,

(2) proteins that act at the level of the mitochondria, including two proapoptotic Bcl2 family members and three inhibitors of antiapoptotic Bcl2 family members, and

(3) a third group of proteins that lead to the generation of reactive oxygen species.

One of the clear results thus far is that no one target of p53-dependent apoptosis can fully account for the loss of death phenotype observed in cells-deficient for p53 (FIG. 3). Thus one of the important questions is why are there so many targets of p53 that can induce cell death? Are all these targets and pathways used each time p53 induces cell death? Applicants believe that there is tissue specificity or signal specificity to the response. It is believed that there is some built-in redundancy in the signaling pathway, because of its importance in suppressing cancer.

In considering the importance of targets of p53 in the cell death response, it has sometimes been argued that many targets may be “artifacts,” (of overexpression of p53). Such generalizations risk overlooking potentially very important targets, including ones that may ultimately prove to be key targets for drug development. It is clear that genes containing classical p53 DNA-binding consensus response elements are excellent candidates for regulation by p53. It also seems somewhat intuitively obvious that cells which expend energy and utilize building blocks to upregulate gene expression would not do so for no reason or in a wasteful manner. It is therefore expected that if a cell upregulates a gene in response to p53 stabilization or overexpression, such a gene is involved in the death response. However, such a gene may be necessary but not sufficient, or it may not even be required under certain conditions. p53 is a transcription factor that has had plenty of time to evolve from the Drosophila m. homologue to respond to a variety of signals and to potentially be subject to a number of modifications or cellular interactions that modulate its target gene selectivity. Applicants have found that “target gene selectivity” is an excellent model to explain the divergent phenotypes (e.g. arrest or apoptosis) observed in response to p53 activation. The property of p53 as a transcription factor allows it to upregulate the necessary targets to achieve a desired response. Based on the degeneracy of the p53 response element, there are believed to be at a minimum several hundred bona fide p53 effector genes. Moreover, in considering targets of p53 that are directly upregulated, the most attractive effectors are ones with a plausible mechanism of action, i.e. targets which can be linked to the caspase machinery of cell death.

How does p53 cause cell death? There is very good evidence that caspase 9 and APAF1 are crucial late downstream effectors of the p53-regulated cell death response (Soengas M S, et al., Apaf-1 and caspase-9 in p53-dependent apoptosis and tumor inhibition, Science 284:156-159 (1999)). Such studies suggest that at least in some cell types, e.g. embryonic fibroblasts that also express ElA and ras, the p53-dependent death signal ultimately travels through the mitochondria leading to formation of the apoptosome. Recent studies have identified certain melanoma tumor cell lines with reduced expression levels of APAF1, and showed that the reexpression of APAF1 by the use of 5′-azacytidine could resensitize cells to the apoptotic effects of DNA damaging chemotherapeutic agents (Soengas M S, et al., supra). APAF1 has also been recently described as a p53 target gene. There is also good evidence that p53 directly controls death-inducing genes that can directly promote cytochrome C release (proapoptotic Bcl2 family members Bax and Bak, and inhibitors of anti-apoptotic Bcl2 family members Noxa, p53AIP1 and PUMA), as well as death inducing genes of the death receptor class (Fas and KILLER/DR5) (Vogelstein B, et al., supra; Wu G S, et al., KILLER/DR5, a novel DNA-damage inducible death receptor gene, links the p53-tumor suppressor to caspase activation and apoptotic death, Adv. Exp. Med. Biol. 465:143-151 (2000); El-Deiry W S, supra; Oda K, et al., p53AIP1, a potential mediator of p53-dependent apoptosis, and its regulation by ser-46-phosphorylated p53, Cell 102:849-862 (2000); Zhao R B, et al., Analysis of p53-regulated gene expression patterns using oligonucleotide arrays, Genes Dev. 14:981-993 (2000); Oda E, et al., Noxa, a BH3-only member of the Bcl-2 family and candidate mediator of p53-induced apoptosis, Science 288:1053-1058 (2000); Yu J, et al., PUMA induces rapid apoptosis of colorectal cancer cells, Mol. Cell 7:673-682 (2001); Nakano K, et al., PUMA, a novel proapoptotic gene, is induced by p53, Mol. Cell 7:683-694 (2001); Lindsten T, et al., The combined functions of proapoptotic Bcl 2 family members Bak and Bax are essential for normal development of multiple tissues, Mol. Cell 6: 1389-1399 (2000); and Lin Y P, et al., Pidd, a new death-domain-containing protein, is induced by p53 and promotes apoptosis, Nature Genet. 26:122-125 (2000)).

A p53-dependent increase in death receptor expression is potentially a mechanism by which cells may be more effectively killed by the immune system, and it appears to be a mechanism for enhanced killing of cancer cells exposed to death-inducing ligands plus cytotoxic agents or radiation (Wu G S, et al., KILLER/DR5, a novel DNA-damage inducible death receptor gene, links the p53-tumor suppressor to caspase activation and apoptotic death, Adv. Exp. Med. Biol. 465:143-151 (2000); Chinnaiyan A M, et al., Combined effect of tumor necrosis factor-related apoptosis-inducing ligand and ionizing radiation in breast cancer therapy, Proc. Natl. Acad. Sci. USA 97:1754-1759 (2000); Nagane M, et al., The potential of TRAIL for cancer chemotherapy, Apoptosis 6:191-197 (2001); Cuello M, et al., Synergistic induction of apoptosis by the combination of TRAIL and chemotherapy in chemoresistant ovarian cancer cells, Gynecol. Oncol. 81:380-390 (2001); Mizutani Y, et al., Enhanced sensitivity of bladder cancer cells to tumor necrosis factor related apoptosis inducing ligand mediated apoptosis by cisplatin and carboplatin, J. Urol. 165:263-270 (2001); Nagane M, et al., Increased death receptor 5 expression by chemotherapeutic agents in human gliomas causes synergistic cytotoxicity with tumor necrosis factor-related apoptosis-inducing ligand in vitro and in vivo, Cancer Res. 60: 847-853 (2000); and Keane M M, et al., Chemotherapy augments TRAIL-induced apoptosis in breast cell lines, Cancer Res. 59: 734-741 (1999)).

Activation of death receptors such as Fas or KILLER/DR5 leads to recruitment of the adaptor FADD and the initiator caspases 8 and 10 to the cell membrane (Ashkenazi A, et al., Death receptors: Signaling and modulation, Science 281:1305-1308 (1998) and Hengartner M O, The biochemistry of apoptosis, Nature 407:770-776 (2000)). Through induced proximity, caspases 8 and 10 become activated and can then trigger the caspase cascade. Downstream of caspase 8 is Bid, which can be cleaved and translocate to the mitochondria to trigger cytochrome C release through interactions with Bak or Bax. There is evidence that both caspases 8 and 9 are cleaved in response to p53 and that p53-dependent apoptosis can be blocked by inhibitors of caspase 8 or caspase 9, i.e. cFLIP or C8I, and BclXL or C9I, respectively (Ozoren N, et al., The caspase 9 inhibitor Z-LEHD-FMK protects human liver cells while permitting death of cancer cells exposed to tumor necrosis factor-related apoptosis-inducing ligand, Cancer Res. 60:6259-6265 (2000); Burns T F, et al., Tissue specific expression of p53 target genes suggests a key role for KILLER/DR5 in p53-dependent apoptosis in vivo, Oncogene, 20:4601-4612 (2001)). The available evidence thus far suggests that neither Fas nor Bax is required for p53-dependent cell death (Fuchs E J, et al., p53-dependent DNA damage-induced apoptosis requires Fas/APO-1-independent activation of CPP32 beta, Cancer Res. 57:2550-2554 (1997); and Knudson C M, et al., Bax-deficient mice with lymphoid hyperplasia and male germ-cell death, Science 270:96-99 (1995)). However, because they are induced, they may contribute to cell death under such situations. Bax appears to be important for neuronal cell death and the deficiency in cell death is much more pronounced if Bak is also deleted (Lindsten T, et al., The combined functions of proapoptotic Bcl-2 family members Bak and Bax are essential for normal development of multiple tissues, Mol. Cell 6:1389-1399; Cregan S P, et al., Bax-dependent caspase-3 activation is a key determinant in p53-induced apoptosis in neurons, J. Neurosci. 19:7860-7869 (1999); and Wei M C, et al., Proapoptotic BAX and BAK: A requisite gateway to mitochondrial dysfunction and death, Science 292:727-730 (2001)). It is noteworthy that Bax/Bak double-null mice, although deficient in DNA damage-induced cell death, do not develop tumors like p53-null mice.

Utilizing the TRAIL Pathway in p53-targeted and p53-independent Cancer Therapy

Applicants' interest in the TRAIL pathway and the p53 pathway began in about 1997 when the proapoptotic TRAIL receptor KILLER/DR5 was cloned by Applicants from a subtractive hybridization screen as a DNA damage-inducible p53-regulated gene (see Accession No. AF022386; Wu G S, et al., KILLER/DR5 is a DNA damage-inducible p53-regulated death receptor gene, Nature Genet. 17:141-143 (1997); Kastan M, On the TRAIL from p53 to apoptosis?, Nature Genet. 17:130-131 (1997); and Ashkenazi A, et al., Death receptors: Signaling and modulation, Science 281:1305-1308 (1998)). Recent work has identified p53 response elements in the human genomic KILLER/DR5 locus, and the p53-dependent regulation of KILLER/DR5 appears to be conserved in the mouse (Takimoto R, et al., Wild-type p53 transactivates the KILLER/DR5 gene through an intronic sequence-specific DNA-binding site, Oncogene 19:1735-1743 (2000) and Wu G S, et al., Molecular cloning and functional analysis of the mouse homologue of the KILLER/DR5 tumor necrosis factor-related apoptosis-inducing ligand (TRAIL) death receptor, Cancer Res 59:2770-2775 (1999)). TRAIL is of great interest to cancer biologists because of its ability to cause cell death of transformed and cancer cells but not most normal cells (Ashkenazi A, et al., Death receptors: Signaling and modulation, Science 281:1305-1308 (1998)) (FIG. 4).

The resistance of normal cells to the cytotoxic effects of TRAIL appear to be in part mediated by high surface expression of TRAIL decoy receptors that compete for binding to TRAIL and thereby reduce activation of the death signal through the proapoptotic TRAIL receptors (Kim K H, et al., Molecular determinants of response to TRAIL in killing of normal and cancer cells, Clin. Cancer Res. 6:335-346 (2000)). Some normal cells also appear to express high levels of cellular FLIP, the Flice-inhibitory protein that is also overexpressed in some cancer cells (Kim K H, et al., Molecular determinants of response to TRAIL in killing of normal and cancer cells, Clin. Cancer Res. 6:335-346 (2000)). Recent work on elucidating the signaling pathway downstream of TRAIL receptors has provided good evidence for the involvement of the FADD adaptor and caspase 8 as the initiator caspase (Bodmer J L, TRAIL receptor-2 signals apoptosis through FADD and caspase-8, Nature Cell Biol. 2:241-243 (2000)).

A number of studies have evaluated the sensitivity of human tumor cells to the cytotoxic effects of TRAIL. It is clear that not all cancer cells are sensitive to the killing effects of TRAIL (Kim K H, et al., supra). Correlations between loss of death receptor DR4 expression and elevated cellular FLIP expression as independent predictors of TRAIL resistance have been made in some studies (Kim K H, et al., supra). A recent study has identified hypermethylation of caspase 8 as a mechanism of inactivation in neuroblastomas, leading to TRAIL resistance which could be reversed by exposing cells to the demethylating agent 5-azacytidine (Teitz T, et al., Caspase 8 is deleted or silenced preferentially in childhood neuroblastomas with amplification of MYCN, Nature Med. 6:529-535 (2000)). Loss of DR4 expression through homozygous deletion resulting from translocation events was observed in the aneuploid FaDu nasopharyngeal cancer cell line (Ozoren N, et al., Homozygous deletion of the death receptor DR4 gene in a nasopharyngeal cancer cell line is associated with TRAIL resistance, Int. J. Oncol. 16:917-925 (2000)). FaDu cells which are resistant to TRAIL were partially sensitized through exogenous expression of wild-type DR4.

Death receptor DR5 mutations have been described in head and neck, lung, and breast cancer (Pai S I, et al., Rare loss-of-function mutation of a death receptor gene in head and neck cancer, Cancer Res. 58:3513-3518 (1998); Lee S H, et al., Alterations of the DR5/TRAIL receptor 2 gene in non-small cell lung cancers, Cancer Res. 59:5683-5686 (1999); and Shin M S, et al., Mutations of Tumor Necrosis Factor-related Apoptosis-inducing Ligand Receptor 1 (TRAIL-R1) and Receptor 2 (TRAIL R2) genes in metastatic breast cancers, Cancer Res. 61:4942-4946 (2001)). A truncating mutation in the death domain was found in head and neck cancer and a number of mutations in the death domain were observed in lung and breast cancer, leading to loss of function. These studies support the notion that the proapoptotic TRAIL receptors are candidate tumor suppressor genes. Recent work has provided evidence for TRAIL production by endogenous natural killer cells in a pathway stimulated by g-interferon (Takeda K, et al., Involvement of tumor necrosis factor-related apoptosis-inducing ligand in surveillance of tumor metastasis by liver natural killer cells, Nature Med. 7:94-100 (2001)). Blockade of TRAIL through the use of a blocking antibody was shown to lead to substantial effects on increasing tumor xenograft growth consistent with the idea that TRAIL may be a tumor suppressor in vivo. Further support for this pathway is suggested by experiments showing that the TRAIL receptor KILLER/DR5 is also upregulated in g-IFN-treated cells (Meng R D, et al., p53-Independent upregulation of KILLER/DR5 TRAIL receptor expression by glucocorticoids and interferon-gamma, Exp. Cell Res. 262:154-169 (2001)). Additional support for the idea that TRAIL receptors are involved in suppressing tumors is suggested by a recent study that has identified a higher frequency of certain polymorphisms in the DR4 gene in the germline of individuals who developed cancer as compared to individuals without cancer (Fisher M J, et al., Nucleotide substitution in the ectodomain of TRAIL receptor DR4 is associated with lung cancer and head and neck cancer, Clin. Cancer Res. 7:1688-1697 (2001)).

Preclinical studies utilizing TRAIL have provided in vivo evidence for exogenous recombinant TRAIL efficacy in suppressing tumor growth (Walczak H, et al., Tumoricidal activity of tumor necrosis factor related apoptosis-inducing ligand in vivo, Nature Med. 5:157-163 (1999)). Moreover, it appears that TRAIL efficacy can be increased in wild-type p53 expressing cells through the combined use of DNA damaging chemotherapeutics or ionizing radiation (Ashkenazi A, supra; Chinnaiyan A M, et al., Combined effect of tumor necrosis factor-related apoptosis-inducing ligand and ionizing radiation in breast cancer therapy, Proc. Natl. Acad. Sci. USA 97:1754-1759 (2000); Nagane M, et al., The potential of TRAIL for cancer chemotherapy, Apoptosis 6: 191-197 (2001); Cuello M, et al., Synergistic induction of apoptosis by the combination of TRAIL and chemotherapy in chemoresistant ovarian cancer cells, Gynecol. Oncol. 81:380-390 (2001); Mizutani Y, et al., Enhanced sensitivity of bladder cancer cells to tumor necrosis factor related apoptosis inducing ligand mediated apoptosis by cisplatin and carboplatin, J. Urol. 165:263-270 (2001); Nagane M, et al., Increased death receptor 5 expression by chemotherapeutic agents in human gliomas causes synergistic cytotoxicity with tumor necrosis factor-related apoptosis-inducing ligand in vitro and in vivo, Cancer Res. 60:847-853 (2000); Keane M M, et al., Chemotherapy augments TRAIL-induced apoptosis in breast cell lines, Cancer Res. 59:734-741 (1999)). This is believed to be due to the p53-dependent upregulation of the TRAIL receptor KILLER/DR5 following DNA damage. A direct test of this hypothesis was recently performed by showing inhibition of cell death due to the combination of p53 overexpression (delivered by adenovirus) and TRAIL through the use of a soluble extracellular domain of the KILLER/DR5 receptor to compete with TRAIL binding to the upregulated DR5 (Kim K, et al., Enhanced TRAIL sensitivity by p53 overexpression in human cancer but not normal cell lines, Int. J. Oncol. 18:241-247 (2001)). While it is clear that TRAIL is effective in killing mutant p53-expressing cancer cells and as such is a promising agent, the increased efficacy realized by activation of the p53 pathway is expected to have therapeutic utility versus certain tumors. Other strategies under development include the combination of TRAIL with g-IFN (Takeda K, et al., Involvement of tumor necrosis factor-related apoptosis-inducing ligand in surveillance of tumor metastasis by liver natural killer cells, Nature Med. 7:94-100 (2001)), or enhancement of cell killing through cell cycle modulation (Jin Z, et al., Enhanced sensitivity of G1 arrested human cancer cells suggests a novel therapeutic strategy using a combination of Simvastatin and TRAIL, Proc. Amer. Assoc. Cancer Res. 42:438 (2001)).

Some studies have observed that recombinant human TRAIL may be toxic to some normal human or mouse cells (Jo M, et al., Apoptosis induced in normal human hepatocytes by tumor necrosis factor-related apoptosis-inducing ligand, Nature Med. 6:564-567 (2000); Nagata S, Steering anti-cancer drugs away from the TRAIL, Nature Med. 6:502-503 (2000)). However, recent studies have suggested that not all preparations of TRAIL are equally toxic to normal human cells (Lawrence D, et al., Differential hepatocyte toxicity of recombinant Apo2L/TRAIL versions, Nature Med. 7:383-385 (2001)). It was reported that untagged full-length human TRAIL binds reversibly to liver cells whereas tagged-TRAIL which is crosslinked using an antibody binds irreversibly. The kinetics and molecular basis for these differences are still under investigation, as well as the ultimate impact of reversible versus irreversible TRAIL binding on cancer cell death in terms of efficacy. One study has provided evidence for cell-type specific effects of TRAIL in killing normal or cancer cells (Ozoren N, et al., The caspase 9 inhibitor Z-LEHD-FMK protects human liver cells while permitting death of cancer cells exposed to tumor necrosis factor-related apoptosis-inducing ligand, Cancer Res. 60:6259-6265 (2000)).

The Present Invention

Knowledge of the emerging pathways of cell death downstream of the p53 tumor suppressor and the TRAIL death-inducing ligand suggests ways to improve cancer therapeutic design. There are signals transduced by p53 to multiple apoptotic effectors due to the apparent importance of apoptosis in suppressing tumors. There is evidence for cytoplasmic as well as mitochondrial activation of caspases downstream of p53, although in some cell lineages the signal ultimately involves the mitochondria. Understanding the cell death pathways induced by p53 activation or engagement of death receptors by the TRAIL ligand is leading to novel strategies for therapeutic design in cancer. In particular, it is expected that the emerging complex phenotypes involving cell- and tissue-specific signaling events will give rise to techniques which shift the balance in favor of cancer cell death and against normal cell death to widen the therapeutic window. The p53 and TRAIL signaling pathways represent ideal targets for therapeutic development because of their relevance to cancer cell apoptosis and the numerous potential ways in which to intervene and modulate. Ultimately, it is expected that cancer therapy will become individualized at the molecular level, perhaps through the use of microarrays (Ricci M S, et al., Novel strategies for therapeutic design in molecular oncology using gene expression profiles, Curr. Opin. Mol. Ther. 2:682-690 (2000)), and such approaches will identify additional specific targets in the death pathway whose induction or inhibition would improve therapeutic outcome.

In developing a successful cancer therapeutic strategy, it becomes useful to incorporate ways to increase death of cancer cells while keeping normal cells alive (FIG. 1). A problem in treating cancer cells is that cancer chemotherapy and radiotherapy, although often effective, are well known to have side-effects which can be severe and limit therapy. Thus, even established regimens can be improved and it is useful to understand the basis of the toxicity of the therapeutic agents, in addition to appreciating their potential efficacy. Strategies that enhance death of cancer cells, while having low toxicity towards normal cells, are needed in order to deliver successful therapy with an acceptable therapeutic window.

In the methods of the present invention, Applicants have identified specific targets in the apoptosis pathway whose induction or inhibition would improve therapeutic outcome.

### SUMMARY OF THE INVENTION

The present invention relates to a method for modulating cellular apoptosis in an animal by regulating the quantity of functional KILLER/DR5 receptor protein molecules in the target cell(s) of said animal.

The present invention further relates to a method for treating a disease or condition in an animal, which comprises:

(a) introducing one or more copies of an isolated exogenous wild-type KILLER/DR5 receptor nucleic acid sequence capable of being expressed, into target cell(s) of said animal; and

(b) treating said animal by administering a therapy selected from the group consisting of radiotherapy and chemotherapy.

The present invention further relates to a method for treating a disease or condition in an animal, which comprises:

(a) introducing one or more copies of an isolated exogenous loss-of-function KILLER/DR5 receptor nucleic acid sequence capable of being expressed, into a target spleen cell or small intestine cell of said animal; and

(b) treatment of said animal with a therapy selected from the group consisting of radiotherapy and chemotherapy.

The present invention further relates to an isolated nucleic acid sequence for the KILLER/DR5 receptor protein, selected from the group consisting of a wild-type KILLER/DR5 receptor nucleic acid sequence bearing one or more point mutations at amino acid(s) 325, 331, 334, 336, 338, 339, 340, 343, 351, and/or 360.

Finally, the present invention relates to a pharmaceutical composition comprising:

(a) a therapeutically acceptable amount of an isolated nucleic acid sequence for the KILLER/DR5 receptor protein, selected from the group consisting of a wild-type KILLER/DR5 receptor nucleic acid sequence bearing one or more point mutations at amino acid(s) 325, 331, 334, 336, 338, 339, 340, 343, 351, and/or 360; and

(b) a pharmaceutically acceptable carrier.

### BRIEF DESCRIPTION OF THE DRAWINGS

FIG. 1 is a drawing which depicts a desirable strategy to achieve therapeutic benefit in cancer therapy.

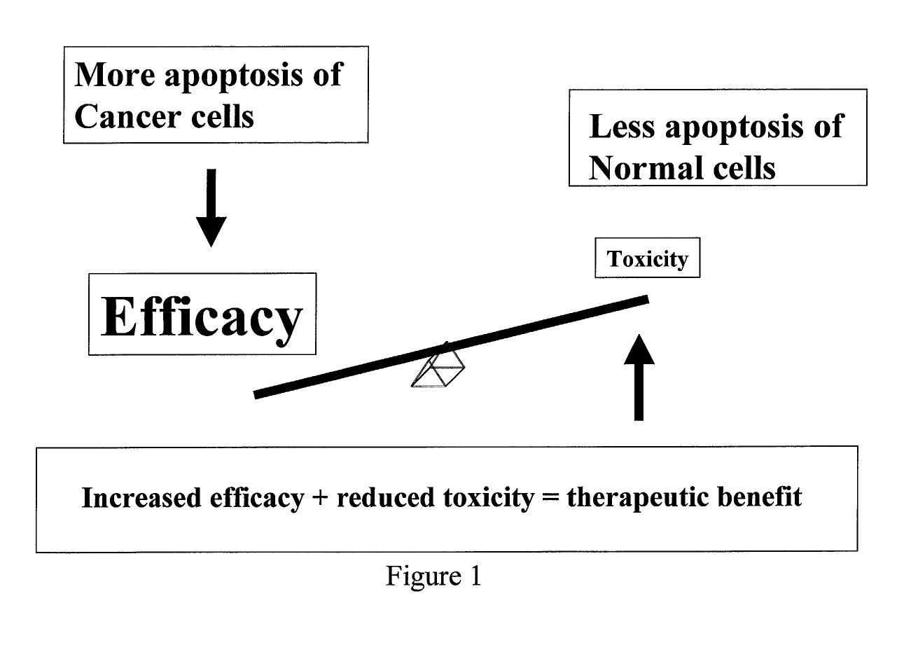

FIG. 2 is a drawing which depicts cell cycle arrest mediated by p53 targets and other DNA damage checkpoint proteins.

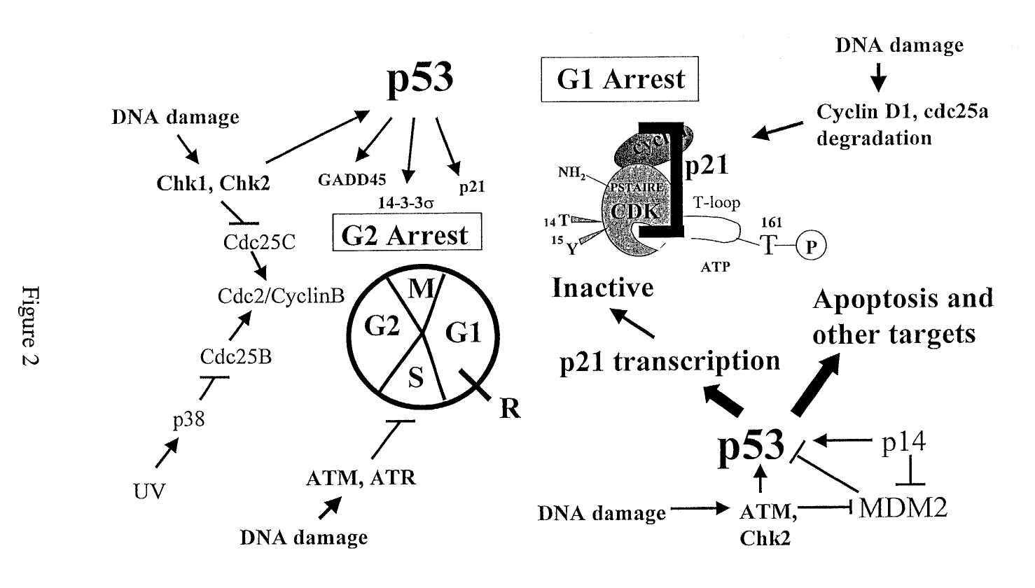

FIG. 3 is a drawing which depicts the pathways downstream of p53 that can cause cell death.

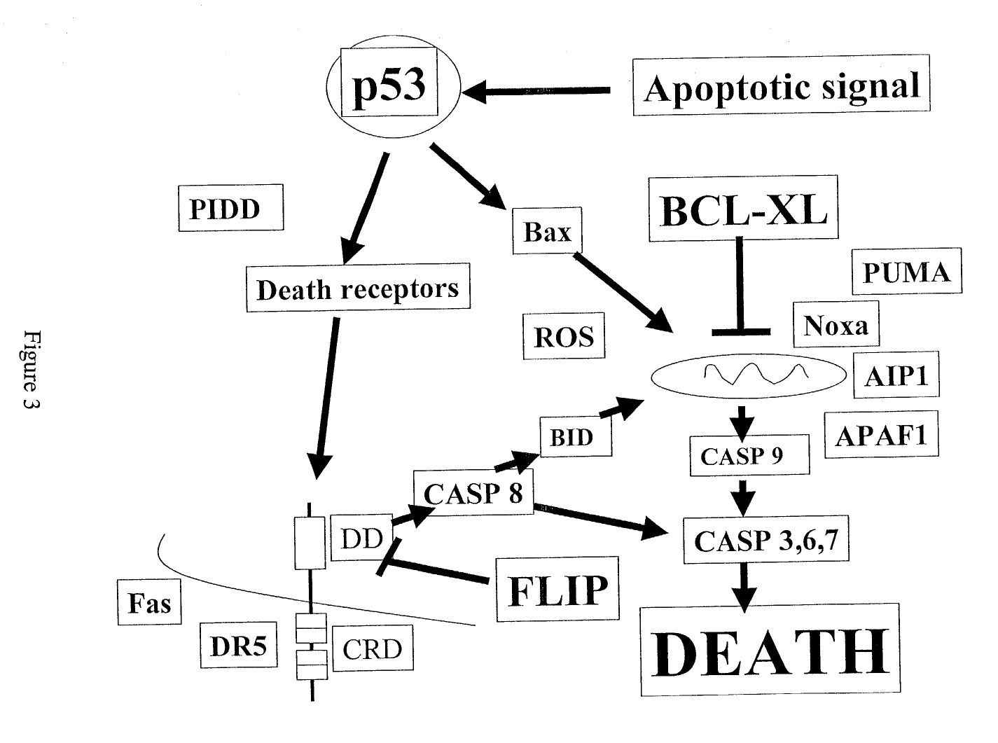

FIG. 4 is a drawing which depicts sensitivity and resistance to TRAIL-induced cell death; chemotherapy or radiation can be combined with TRAIL to achieve synergistic cell killing, in part through p53-dependent upregulation of KILLER/DR5 expression.

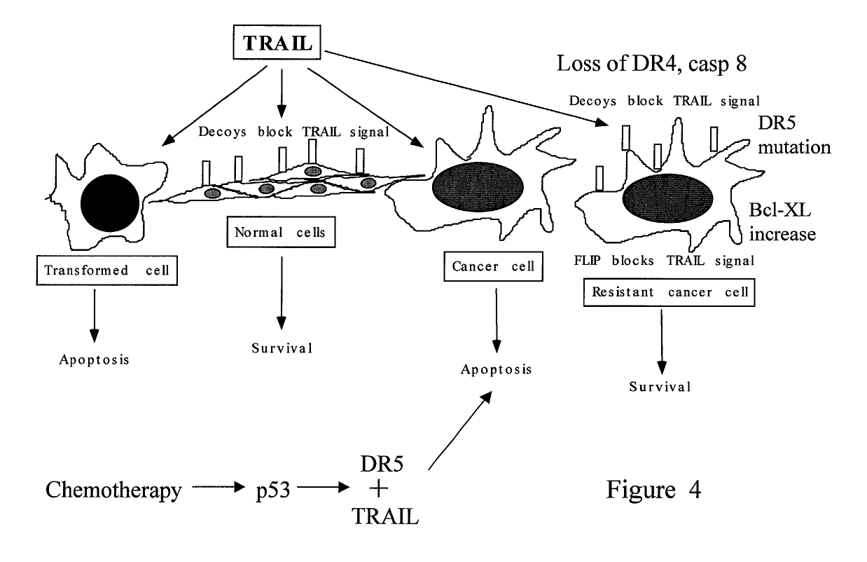

FIG. 5 depicts percentage of transfected cells which express activated caspase 3. FIG. 5(a) is a photograph which depicts Western blot analysis for PARP cleavage after adenovirus infection. FIG. 5(b) is a chart which depicts Western blot analysis of caspase 8 and 9 cleavage after adenovirus infection. FIG. 5(c) is a photograph which depicts FACS analysis for percentage of cells with a sub-G₁ content after adenoviral infection. FIG. 5(d) is a chart which depicts FACS analysis for active caspase 3 in adrenal carcinoma cell line SW13 after transfection with p53.

FIG. 6 is a photograph which depicts Western analysis of PARP cleavage after treatment in p53+/+ and p53−/− animals, showing that ionizing radiation induces a p53-dependent apoptosis in the spleen and thymus. Left panel: spleen right panel: Thymus.

FIG. 7 is a graph which depicts expression of p53 target genes in the spleen after irradiation or dexamethasone treatment in p53+/+ and p53−/− animals. FIG. 7(a) depicts p21 mRNA expression in p53+/+ animals and p53−/− animals after irradiation or dexamethasone treatment. FIG. 7(b) depicts mRNA levels of four p53 target genes. FIG. 7(c) is a photograph which depicts Western blot analysis for bax and p21 after irradiation or dexamethasone treatment. FIG. 7(d) depicts oligonucleotide microarray analysis of total RNA from the spleen of irradiated and untreated p53+/+ and p53−/− animals.

FIG. 8 is a chart which depicts expression of p53 target genes in the thymus after irradiation or dexamethasone treatment in p53+/+ and p53−/− animals. FIG. 8(a) depicts p21 mRNA expression in p53+/+ animals and p53−/−animals after irradiation or dexamethasone treatment. FIG. 8(b) depicts mRNA levels of four p53 target genes. FIG. 8(c) is a photograph which depicts Western blot analysis for bax and p21 after irradiation. FIG. 8(d) depicts oligonucleotide microarray analysis of total RNA from the thymus of irradiated and untreated p53+/+ and p53−/− animals.

FIG. 9 is a chart which depicts expression of p53 target genes in the small intestine after irradiation or dexamethasone treatment in p53+/+ and p53−/− animals. FIG. 9(a) depicts p21 mRNA expression in p53+/+ animals and p53−/− animals after irradiation or dexamethasone treatment. FIG. 9(b) depicts mRNA levels of four p53 target genes. FIG. 9(c) depicts Western blot analysis for bax and p21 after irradiation.

FIG. 10 is a drawing which depicts alignment of DD from TNF receptor superfamily members.

FIG. 11 is a photograph which depicts protein expression of wild-type and alanine mutant KILLER/DR5 constructs.

FIG. 12 is a graph which depicts flow cytometry/GFP-spectrin based assay to assess sub-G₁ peak following transient transfection of wild type and alanine mutant KILLER/DR5 constructs.

FIG. 13 photograph which depicts PARP cleavage following transient transfection of wild-type (W.T.) and alanine mutant KILLER/DR5 constructs.

FIG. 14 is a photograph which depicts protein expression and PARP cleavage induction by tumor-derived KILLER/DR5 mutants.

FIG. 15(A) is photograph which depicts TRAIL DISC immunoprecipitation of transfected 293 cells. FIG. 15(B) is photograph which depicts TRAIL DISC immunoprecipitation of panel of total and partial loss-of-function alanine mutants. FIG. 15(C) is photograph which depicts TRAIL DISC immunoprecipitation of the alanine versus tumor loss-of-function mutants.

### DETAILED DESCRIPTION OF THE INVENTION

### Definitions

“Toxicity response” refers to the response to a toxic therapy or treatment such as chemotherapy or radiotherapy.

“Isolated nucleic acid sequence” refers to a nucleic acid sequence which has been purified to at least about 95% homogeneity. This definition includes nucleic acid sequences that hybridize under stringent hybridization conditions with a gene, such as a cDNA molecule. This definition further includes a genomic sequence of interest comprising a nucleic acid sequence between the initiation codon and the stop codon, including all of the introns that are normally present in a native chromosome. It may further include the 3′ and 51′ untranslated regions found in a mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kb or smaller; and substantially free of flanking chromosomal sequence. The genomic DNA flanking the coding region, either 3′ or 5′, or internal regulatory sequences as sometimes found in introns, contains sequences required for proper tissue and stage specific expression.

“Modulate,” in the context of the present invention, refers to an increase or decrease in the quantity of functional protein in a cell. Such regulation may be accomplished by introducing either a wild-type or mutant nucleic acid sequence capable of being expressed, or by administering a therapeutically effect amount of a functional or defective protein.

“Wild-type nucleic acid sequence” refers to the nucleic acid sequence(s) found in subjects having normal function in the protein transcribed from the nucleic acid sequence(s). It is to be understood that some variation in wild-type nucleic acid sequence is to be expected, and the salient feature is normal protein function. “Wild-type” nucleic acid sequence is to be distinguished from nucleic acid sequence(s) transcribing defective or loss-of-function proteins.

A nucleic acid sequence “capable of being expressed” refers to a nucleic acid sequence which, when introduced into a target cell, forms a complete transcription complex. The resulting complete transcription complex may comprise the required elements as introduced with the exogenous nucleic acid sequence, as provided by the target cell, or a combination of introduced and existing elements.

“TRAIL” refers to Tumor Necrosis Factor-Related Apoptosis Inducing Ligand.

“Exogenous” refers to a substance originating outside of an organism.

“Endogenous” refers to a substance originating within an organism.

“Loss-of-function mutant receptor proteins” refers to proteins which exhibit a diminished function in relation to wild-type protein function as a result of one or more point mutation(s) in the nucleic acid sequence(s) coding for said protein.

“Isolated protein” refers to a protein which can be obtained from its natural source, can be produced using recombinant DNA technology, or can be produced by chemical synthesis.

“Active agent” refers to wild-type or mutant nucleic acid sequence(s), functional or defective proteins, and therapeutic agents, including, but not limited to chemotherapeutic agents, radiotherapy, ablation or other therapeutic hormones, antineoplastic agents, monoclonal antibodies useful against cancer cells, and angiogenesis inhibitors.

“Effecting” refers to the process of producing an effect on biological activity, function, health, or condition of an organism in which such biological activity, function, health, or condition is maintained, enhanced, diminished, or treated in a manner which is consistent with the general health and well-being of the organism.

“Enhancing” the biological activity, function, health, or condition of an organism refers to the process of augmenting, fortifying, strengthening, or improving.

“Pharmaceutically acceptable salt, ester, or solvate” refers to a salt, ester, or solvate of a subject compound which possesses the desired pharmacological activity and which is neither biologically nor otherwise undesirable. A salt, ester, or solvate can be formed with inorganic acids such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, gluconate, glycerophosphate, hemisulfate, heptanoate, hexanoate, hydrochloride, hydrobromide, hydroiodide, 2-hydroxyethanesulfonate, lactate, maleate, methanesulfonate, naphthylate, 2-naphthalenesulfonate, nicotinate, oxalate, sulfate, thiocyanate, tosylate and undecanoate. Examples of base salts, esters, or solvates include ammonium salts; alkali metal salts, such as sodium and potassium salts; alkaline earth metal salts, such as calcium and magnesium salts; salts with organic bases, such as dicyclohexylamine salts; N-methyl-D-glucamine; and salts with amino acids, such as arginine, lysine, and so forth. Also, the basic nitrogen-containing groups can be quarternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; aralkyl halides, such as benzyl and phenethyl bromides; and others. Water or oil-soluble or dispersible products are thereby obtained.

### Modulation of Cellular Apoptosis

The present invention relates to the use of wild-type and loss-of-function TRAIL receptor KILLER/DR5 nucleic acid sequences and proteins in modulating apoptosis (programed cell death). In particular, the proteins of the invention can be used in the induction of apoptosis in tumor cells. The proteins according to the invention can also be used in the elimination of other target undesired cell populations. The invention further relates to the use of mutant TRAIL receptor KILLER/DR5 nucleic acid sequences and proteins in reducing cellular sensitivity to induction of apoptosis by the downregulation of apoptosis in healthy cells.

The present invention, as exemplified, relates to a preferential, tissue-specific regulation of KILLER/DR5 in spleen and gastrointestinal cells. Such tissue-specific protein expression is expected to guide novel drug development strategies. Specifically, it would be expected that, if a particular effector of p53-dependent apoptosis is favored in a given tissue, targeting its inhibition would reduce toxicity in response to chemo- or radiotherapy. Such strategies are exemplified in the present invention, in which specific determinants of KILLER/DR5-induced death signaling and their loss in tumors is explored.

The idea that blocking the p53 response to reduce toxicity is not novel (Merritt A J, et al., The role of p53 in spontaneous and radiation-induced apoptosis in the gastrointestinal tract of normal and p53-deficient mice, Cancer Res. 54:614-617 (1994)). The novelty of the tissue-specific p53 response of the present invention is to block specific targets favored in a particular tissue that is highly susceptible to toxicity. One of the targets of the present invention is p53-favored KILLER/DR5 in the gut, which is highly sensitive to radiation. It is expected that blocking KILLER/DR5 will reduce toxicity in such sensitive tissues.

### Methods and Compositions for Modulating Apoptosis

The present invention relates to a method for modulating cellular apoptosis in an animal by regulating the quantity of functional KILLER/DR5 receptor protein molecules in the target cell(s) of said animal. A preferred feature of the invention is that the regulation of said protein increases the difference in toxicity response between target cells such as cancer cells and non-target cells such as healthy cells, in response to a therapy or treatment; the regulation of the protein produces either an increase or decrease in functional KILLER/DR5 receptor protein molecules per target cell, depending upon whether the desired effect is upregualtion or downregulation of apoptosis. In a particularly preferred embodiment, said cancer cells are spleen cancer cells or gastrointestinal cancer cells.

In one preferred embodiment, said method further comprises administering to said animal a recombinant viral vector comprising a nucleic acid sequence encoding a wild-type KILLER/DR5 receptor protein or a nucleic acid sequence encoding a loss-of-function mutant KILLER/DR5 receptor protein, said sequence operatively linked to regulatory sequence(s) directing expression of said receptor protein in the target cell(s) of said animal. In a more preferred embodiment, said isolated exogenous mutated KILLER/DR5 receptor nucleic acid sequence is selected from the group consisting of a wild-type KILLER/DR5 receptor nucleic acid sequence bearing one or more point mutations at amino acid(s) 325, 331, 334, 336, 338, 339, 340, 343, 351, and/or 360.

In another preferred embodiment, said method further comprises administering to said animal a wild-type KILLER/DR5 receptor protein or a loss-of-function mutant KILLER/DR5 receptor protein.

The present invention further relates to a method for treating a disease or condition in an animal, which comprises:

(a) introducing one or more copies of an isolated exogenous wild-type KILLER/DR5 receptor nucleic acid sequence capable of being expressed, into target cell(s) of said animal; and

(b) treating said animal by administering a therapy selected from the group consisting of radiotherapy and chemotherapy.

Said nucleic acid sequence and said therapy are optionally administered simultaneously or sequentially.

In a preferred embodiment, the disease is cancer.

The present invention further relates to a method for treating a disease or condition in an animal, which comprises:

(a) introducing one or more copies of an isolated exogenous loss-of-function KILLER/DR5 receptor nucleic acid sequence capable of being expressed, into a target spleen cell or small intestine cell of said animal; and

(b) treatment of said animal with a therapy selected from the group consisting of radiotherapy and chemotherapy.

Said nucleic acid sequence and said therapy are optionally administered simultaneously or sequentially.

In a preferred embodiment, the disease is cancer.

The present invention further relates to an isolated nucleic acid sequence for the KILLER/DR5 receptor protein, selected from the group consisting of a wild-type KILLER/DR5 receptor nucleic acid sequence bearing one or more point mutations at amino acid(s) 325, 331, 334, 336, 338, 339, 340, 343, 351, and/or 360.

Finally, the present invention relates to a pharmaceutical composition comprising:

(a) a therapeutically acceptable amount of an isolated nucleic acid sequence for the KILLER/DR5 receptor protein, selected from the group consisting of a wild-type KILLER/DR5 receptor nucleic acid sequence bearing one or more point mutations at amino acid(s) 325, 331, 334, 336, 338, 339, 340, 343, 351, and/or 360; and

(b) a pharmaceutically acceptable carrier.

In another preferred embodiment of the invention, the therapies of the present invention are used in combination with one or more other therapies or therapeutic agents, as described further herein. Such therapies and agents may be chemotherapeutic agents, radiotherapy, ablation or other therapeutic hormones, antineoplastic agents, monoclonal antibodies useful against cancer cells, and angiogenesis inhibitors. The following discussion highlights some agents in this respect, which are illustrative, not limiting. A wide variety of other effective active agents also may be used.

Chemotherapy

It is expected that the combination of chemotherapy with therapies of the present invention will be synergistic. Among antineoplastic and anticancer agents that may be used in combination with the invention 5-fluorouracil, vinblastine sulfate, extramustine phosphate, suramin and strontium-89 are preferred. Additional examples of chemotherapeutic agents include the compounds of the Table below.

| CHEMOTHERAPEUTIC AGENT | MEDIAN DOSAGE |
|---|---|
| Asparaginase | 10,000 units |
| Bleomycin Sulfate | 15 units |
| Carboplatin | 50-450 mg. |
| Carmustine | 100 mg. |
| Cisplatin | 10-50 mg. |
| Cladribine | 10 mg. |
| Cyclophosphamide | 100 mg.-2 gm. |
| (lyophilized) | |
| Cyclophosphamide (non- | 100 mg.-2 gm. |
| lyophilized) | |
| Cytarabine (lyophilized | 100 mg.-2 gm. |
| powder) | |
| Dacarbazine | 100 mg.-200 mg. |
| Dactinomycin | 0.5 mg. |
| Daunorubicin | 20 mg. |
| Diethyistilbestrol | 250 mg. |
| Doxorubicin | 10-150 mg. |
| Etidronate | 300 mg. |
| Etoposide | 100 mg. |
| Floxuridine | 500 mg. |
| Fludarabine Phosphate | 50 mg. |
| Fluorouracil | 500 mg.-5 gm. |
| Goserelin | 3.6 mg. |
| Granisetron Hydrochloride | 1 mg. |
| Idarubicin | 5-10 mg. |
| Ifosfamide | 1-3 gm. |
| Leucovorin Calcium | 50-350 mg. |
| Leuprolide | 3.75-7.5 mg. |
| Mechlorethamine | 10 mg. |
| Medroxyprogesterone | 1 gm. |
| Melphalan | 50 gm. |
| Methotrexate | 20 mg.-1 gm. |
| Mitomycin | 5-40 mg. |
| Mitoxantrone | 20-30 mg. |
| Ondansetron Hydrochloride | 40 mg. |
| Paclitaxel | 30 mg. |
| Pamidronate Disodium | 30-*90 mg. |
| Pegaspargase | 750 units |
| Plicamycin | 2,500 mcgm. |
| Streptozocin | 1 gm. |
| Thiotepa | 15 mg. |
| Teniposide | 50 mg. |
| Vinblastine | 10 mg. |
| Vincristine | 1-5 mg. |
| Aldesleukin | 22 million units |
| Epoetin Alfa | 2,000-10,000 units |
| Filgrastim | 300-480 mcgm. |
| Immune Globulin | 500 mg.-10 gm. |
| Interferon Alpha-2a | 3-36 million units |
| Interferon Alpha-2b | 3-50 million units |
| Levamisole | 50 mg. |
| Octreotide | 1,000-5,000 mcgm. |
| Sargramostim | 250-500 mcgm. |

Immunotherapy

The present invention may be used in conjunction with immunotherapies, as well as other treatment modalities. Not only may the methods and compositions herein disclosed be used with the increasing variety of immunological reagents now being tested or used to treat cancer, but it also may be used with those that come into practice in the future. The present invention thus may be used with immunotherapies based on polyclonal or monoclonal antibody-derived reagents, for instance. Monoclonal antibody-based reagents are among those most highly preferred in this regard. Such reagents are well known and are described in, for instance, Ritter MONOCLONAL ANTIBODIES—PRODUCTION, ENGINEERING AND CLINICAL APPLICATIONS, Ritter et al., Eds., Cambridge University Press, Cambridge, UK (1995), which is incorporated by reference herein in its entirety. Radiolabeled monoclonal antibodies for cancer therapy, in particular, also are well known and are described in, for instance, CANCER THERAPY WITH RADIOLABELED ANTIBODIES, D. M. Goldenberg, Ed., CRC Press, Boca Raton, Fla. (1995), which is incorporated by reference herein in its entirety.

Cryotherapy

Cryotherapy has been applied to the treatment of some cancer cells. Methods and compositions of the present invention also could be used in conjunction with an effective therapy of this type.

Hormonal Therapy

Among hormones which may be used in combination with the present invention diethylstilbestrol (DES), leuprolide, flutamide, cyproterone acetate, ketoconazole and amino glutethimide are preferred.

### Route(s) of Administration

In a preferred embodiment, isolated TRAIL receptor KILLER/DR5 DNA is inserted into an appropriate vector readily identifiable by one of ordinary skill in the art and described further herein and in the documents incorporated by reference herein. The vector is introduced into the host by methods readily identifiable by one of ordinary skill in the art and described further herein and in the documents incorporated by reference herein.

Alternate route(s) of administration of the compositions of the present invention are well known to those skilled in the art (see, for example, “Remington's Pharmaceutical Sciences”, 18th Edition, Chapter 86, pp. 1581-1592, Mack Publishing Company, 1990). The compositions may be administered orally, parenterally, by inhalation spray, topically, rectally, nasally, buccally, vaginally, or via an implanted reservoir in dosage formulations containing conventional non-toxic pharmaceutically-acceptable carriers, adjuvants, and vehicles. The term parenteral as used herein includes subcutaneous, intravenous, intramuscular, intraperitoneally, intrathecally, intraventricularly, intrasternal, and intracranial injection or infusion techniques.

The compounds and compositions may be administered in the form of sterile injectable preparations, for example, as sterile injectable aqueous or oleaginous suspensions. These suspensions, may be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile, fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

Additionally, the compounds and compositions may be administered orally in the form of capsules, tablets, aqueous suspensions, or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening, flavoring, coloring agents, or combinations thereof. Delivery in an enterically coated tablet, caplet, or capsule, to further enhance stability and provide release in the intestinal tract to improve absorption, is the best mode of administration currently contemplated.

The compounds and compositions may also be administered rectally in the form of suppositories. These compositions can be prepared by mixing the drug with a suitable non-irritating excipient which is solid at room temperature, but liquid at rectal temperature and, therefore, will melt in the rectum to release the drug. Such materials include cocoa butter, beeswax, and polyethylene glycols.

Furthermore, the compounds may be administered topically, especially when the conditions addressed for treatment involve areas or organs readily accessible by topical application, including the lower intestinal tract. Suitable topical formulations can be readily prepared for such areas or organs. For example, topical application to the lower intestinal tract can be effected in a rectal suppository formulations (see above) or in suitable enema formulations.

It is envisioned that the continuous administration or sustained delivery of the compounds and compositions of the present invention may be advantageous for a given condition. While continuous administration may be accomplished via a mechanical means, such as with an infusion pump, it is contemplated that other modes of continuous or near continuous administration may be practiced. For example, such administration may be by subcutaneous or muscular injections as well as oral pills.

Techniques for formulating a variety of other sustained- or controlled-delivery means, such as liposome carriers, bio-erodible particles or beads and depot injections, are also known to those skilled in the art.

### Dosage

The novel methods of the invention involve administration of a therapeutically effective amount of the active agent indicated above. This effective amount can vary depending upon the physical status of the patient and other factors well known in the art. Moreover, it will be understood that this dosage of active agent can be administered in a single or multiple dosage units to provide the desired therapeutic effect. If desired, other therapeutic agents can be employed in conjunction with those provided by the present invention. The compounds of the invention are preferably delivered to the patient by means which are well known in the art. Solid form pharmaceutical preparations which may be prepared according to the present invention include powders, tablets, dispersible granules, capsules, cachets and suppositories. In general, solid form preparations will comprise from about 5% to about 90% by weight of the active agent.

A solid carrier can be one or more substances which may also act as diluents, flavoring agents, solubilizers, lubricants, suspending agents, binders or tablet disintegrating agents; it can also be encapsulating material. In powders, the carrier is a finely divided solid which is in admixture with the viscous active compound. In tablets, the active compound is mixed with a carrier having the necessary binding properties in suitable proportions and compacted to the shape and size desired. Suitable solid carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating materials as a carrier which may provide a capsule in which the active component (with or without other carriers) is surrounded by carrier, which is thus in association with it. Similarly, cachets are included. Tablets, powders, cachets, and capsules can be used as solid dosage forms suitable for oral administration. If desired for reasons of convenience or patient acceptance, pharmaceutical tablets prepared according to the invention may be provided in chewable form, using techniques well known in the art.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby to solidify.

Liquid form preparations include solutions, suspensions, and emulsions. As an example may be mentioned water or water/propylene glycol solutions for parenteral injection. Liquid preparations can also be formulated in solution in aqueous polyethylene glycol solution. Aqueous solutions suitable for oral use can be prepared by dissolving the active component in water and adding suitable colorants, flavors, stabilizers and thickening agents as desired. Aqueous suspensions suitable for oral use can be made my dispersing the finely divided active component in water with a viscous material, i.e., natural or synthetic gums, resins, methylcellulose, sodium carboxymethylcellulose, and other well known suspending agents. Liquid pharmaceutical preparations may comprise up to 100% by weight of the subject active agent.

Also contemplated as suitable carriers are solid form preparations which are intended to be converted, shortly before use, to liquid form preparations for either oral or parenteral administration. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container. When multiple liquid doses are so prepared, it is preferred to maintain the unused portion of said liquid doses at low temperature (i.e., under refrigeration) in order to retard possible decomposition. The solid form preparations intended to be converted to liquid form may contain, in addition to the active material, flavorants, colorants, stabilizers, buffers, artificial and natural sweeteners, dispersants, thickeners, solubilizing agents, and the like. The liquid utilized for preparing useful liquid form preparations may be water, isotonic water, ethanol, glycerine, propylene glycol, and the like as well as mixtures thereof. Naturally, the liquid utilized will be chosen with regard to the route of administration. For example, liquid preparations containing large amounts of ethanol are not suitable for parenteral use.

The pharmaceutical preparation may also be in a unit dosage form. In such form, the preparation is subdivided into unit doses containing appropriate quantities of the active component. The unit dosage form can be a packaged preparation, the package containing discrete quantities of preparation, for example, packeted tablets, capsules, and powders in vials or ampoules. The unit dosage form can also be a capsule, cachet, or tablet itself or it can be the appropriate number of any of these in packaged form.

The pharmaceutical preparations of the invention may include one or more preservatives well known in the art, such as benzoic acid, sorbic acid, methylparaben, propylparaben and ethylenediaminetetraacetic acid (EDTA) Preservatives are generally present in amounts up to about 1% and preferably from about 0.05 to about 0.5% by weight of the pharmaceutical composition.

Useful buffers for purposes of the invention include citric acid-sodium citrate, phosphoric acid-sodium phosphate, and acetic acid-sodium acetate in amounts up to about 1% and preferably from about 0.05 to about 0.5% by weight of the pharmaceutical composition. Useful suspending agents or thickeners include cellulosics like methylcellulose, carageenans like alginic acid and its derivatives, xanthan gums, gelatin, acacia, and microcrystalline cellulose in amounts up to about 20% and preferably from about 1% to about 15% by weight of the pharmaceutical composition.

Sweeteners which may be employed include those sweeteners, both natural and artificial, well known in the art. Sweetening agents such as monosaccharides, disaccharides and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol and mixtures thereof may be utilized in amounts from about 10% to about 60% and preferably from about 20% to about 50% by weight of the pharmaceutical composition. Water soluble artificial sweeteners such as saccharin and saccharin salts such as sodium or calcium, cyclamate salts, acesulfame-K, aspartame and the like and mixtures thereof may be utilized in amounts from about 0.001% to about 5% by weight of the composition.

Flavorants which may be employed in the pharmaceutical products of the invention include both natural and artificial flavors, and mints such as peppermint, menthol, vanilla, artificial vanilla, chocolate, artificial chocolate, cinnamon, various fruit flavors, both individually and mixed, in amounts from about 0.5% to about 5% by weight of the pharmaceutical composition.

Colorants useful in the present invention include pigments which may be incorporated in amounts of up to about 6% by weight of the composition. A preferred pigment, titanium dioxide, may be incorporated in amounts up to about 1%. Also, the colorants may include other dyes suitable for food, drug and cosmetic applications, known as F.D.&C. dyes and the like. Such dyes are generally present in amounts up to about 0.25% and preferably from about 0.05% to about 0.2% by weight of the pharmaceutical composition. A full recitation of all F.D.&C. and D.&C. dyes and their corresponding chemical structures may be found in the Kirk-Othmer Encyclopedia of Chemical Technology, in Volume 5, at pages 857-884, which text is accordingly incorporated herein by reference.

Useful solubilizers include alcohol, propylene glycol, polyethylene glycol and the like and may be used to solubilize the flavors. Solubilizing agents are generally present in amounts up to about 10%; preferably from about 2% to about 5% by weight of the pharmaceutical composition.

Lubricating agents which may be used when desired in the instant compositions include silicone oils or fluids such as substituted and unsubstituted polysiloxanes, e.g., dimethyl polysiloxane, also known as dimethicone. Other well known lubricating agents may be employed.

It is not expected that compounds of the present invention will display significant adverse interactions with other synthetic or naturally occurring substances. Thus, a compound of the present invention may be administered in combination with other compounds and compositions of the present invention and other active agents as disclosed herein.

The optimal pharmaceutical formulations will be determined by one skilled in the art depending upon considerations such as the route of administration and desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the present therapeutic agents of the invention.

The amount of active ingredient that may be combined with the carrier materials to produce a single dosage form will vary depending upon the host treated and the particular mode of administration. It is understood, however, that a specific dose level for any particular patient will depend upon a variety of factors, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

### EXAMPLES

The following examples are illustrative of the present invention and are not intended to be limitations thereon. Unless otherwise indicated, all percentages are based upon 100% by weight of the final composition.

### Example 1

### Tissue Specific Expression of p53 Target Genes Demonstrates a Key Role for KILLER/DR5 in p53-dependent Apoptosis in Vivo

In this study, the role of both caspase 8 and 9 in p53-dependent apoptosis in two human cancer cell lines and the expression of several p53 target genes involved in apoptosis in an in vivo model of p53-dependent apoptosis was investigated. Both caspase 8 and caspase 9 are cleaved during p53-dependent apoptosis and specific inhibitors of the activity of each, block p53-dependent apoptosis. Furthermore, this study has examined the expression of p53 target genes, including KILLER/DR5, after irradiation of p53-wild-type and-null animals. This study has found a striking tissue-specific expression pattern for these apoptotic target genes. It is expected that KILLER/DR5 plays a key role in mediating p53-dependent apoptosis in vivo, particularly in the spleen and small intestine where its transcriptional induction following γ-irradiation predominates among the known pro-apoptotic targets examined. These results demonstrate the importance of the death receptor pathway in mediating p53-dependent apoptosis.

Materials and Methods

Animals and treatments. Healthy 6-7 week old female p53+/+ and p53−/− animals were used. Two animals in each experimental group received either 0.5 mg/animal of dexamethasone or sterile 1×PBS by single bolus intraperitoneal injection. Total body irradiation was performed using a ¹³⁷Cesium γ-source at a dose rate of 1.532 Gy/min. At 0, 6 and 24 hours, the mice were euthanized using an approved Institutional Animal Care and Use Committee Protocol, which followed recommendations of the Panel on Euthanasia of the American Veterinary Medical Association. Tissues were harvested and snap frozen in liquid nitrogen and kept at −80° C. until used.

Cell culture and adenoviral/retroviral infection. The culture conditions for SW480, HCT 116, and SkBr3 were maintained as previously described (Wu et al., 1997). The adrenal carcinoma cell line, SW13 was a kind gift from R. Sheikhattar (Wistar Institute). Ad-LacZ and Ad-p53 were obtained from B. Vogelstein (Johns Hopkins University) All adenoviruses were propagated, titered, and amplified as previously described (El-Deiry et al., 1993). The retroviral vector pLIB, pLIB-EGFP, pLIB-c-Flip-s, or pLIB-Bcl-XL was cotransfected with pVSVG and pCgP (Dr. Michael Malim, University of Pennsylvania) into 293T cells using the CaPO₄ method. The resulting supernatant was used to infect the SW480 cell line (3×10⁵ cell/well plated 48 hours before infection) at a titer necessary to obtain greater than 90% infectivity as previously described (Fouchier et al., 1997).

Plasmids. The wild type p53 expression vector pCEP4-p53 (El-Deiry et al., 1993) and control vector were generously supplied by Bert Vogelstein (Johns Hopkins University). The pCMVEGFP-Spectrin vector was previously described (Kalejta et al., 1997). The Bcl-XL and c-FLIP-s (short form) (Irmler et al., 1997) genes were amplified from a human placenta retroviral library and cloned into the Notl site of pLIB respectively and fully sequenced prior to use in transfection studies.

Transfections and fluorescence-activated cell sorting (FACS) analysis. Transfections were carried out as previously described (Wu et al., 1997). In brief, before transfection, 5×10⁵ cells/6 cm well were seeded. The cells were transfected with lipofectamine Plus. Four hours after transfections the media was changed and an irreversible caspase inhibitor, Z-IETD-FMK (caspase 8) or Z-LEHD-FMK (caspase 9) was added to the media at a final concentration of 20 μM. Cells were harvested 24 hours after transfection and prepared for detection of active-caspase 3 by flow cytometry as previously described (Ozoren et al., 2000). For adenoviral experiments, cells were harvested 48 hours after infection and fixed and stained with propidium iodide as previously described (Ozoren et al., 2000). Cell sorting was performed.

Western blotting. Total cellular protein was harvested in 1× Laemuli sample buffer and quantitated using the Amido Black Staining method as previously described (Meng et al., 1998). A total of 80 μg of protein per lane was loaded on either a 10% (PARP) or 15% SDS-PAGE gel and electrophoresed and electroblotted as previously described (El-Deiry et al., 1994). The following antibodies were used in these experiments: rabbit polyclonal anti-PARP, mouse monoclonal anti-caspase 8, rabbit polyclonal anti-caspase 9, mouse monoclonal anti-actin, rabbit polyclonal anti-p21, and rabbit polyclonal anti-bax.

Real time quantitative RT-PCR assay for mRNAs of p21, bax, EI24, Fas/APO1, mKILLER/DR5, and GAPDH. RT-PCR assay was conducted. In brief, oligonucleotides (probes) for RT-PCR were labeled with FAM(6-carboxyfluorescin) (p21, bax, EI24, Fas/APO1, and mKILLER/DR5) or VIC (GAPDH) and 3′ prime quenched, TAMRA (6-carboxyl-N,N,N′,N′-tetramethylrhodamine). Whenever possible probe and primer sets were designed over intron/exon boundaries to prevent amplification of genomic DNA. All primer pairs were tested to ensure that the proper size fragment was generated. Total RNA was isolated from individual tissues as previously described (El-Deiry et al., 1993) and 1 μg was used for reverse transcription and amplification.

Total RNA samples were electrophoresed on a MOPS-formaldehyde gel and the pattern of the 28S and 18S ribosomal bands were observed to verify the quality of the total RNA preparation (El-Deiry et al., 1993). A master mix of RT-PCR reagents was prepared and 10 ng of each RT sample was used in the PCR reaction. Each tube contained both a gene probe and primers, and a GAPDH control probe and primer. Each sample was done in quadruplicate. The increase in fluorescence (Δ Rn) was proportional to the concentration of template in the PCR. The PCR cycle number at threshold represents CT. Reactions in which reverse transcriptase was not added to the RT reaction were used to control for genomic contamination. Any contribution of genomic contamination to the CT value for a sample was ignored since the CT value of the (−)RT reaction was at least 10 cycles less than for samples in which reverse transcriptase was added. The standard curve method was used to quantitate amounts of each gene relative to the GAPDH amount in each reaction. Reactions were carried out in 96-well plates using automated sequencing.

Oligonucleotide microarray analysis. Total RNA was isolated from individual tissues as previously described (El-Deiry et al., 1993) and amplified, labeled, and hybridized to the Murine 11K gene chips as previously described (Lockhart et al., 1996; Wodicka et al., 1997). For each RNA sample the average intensity of all genes on the chip was adjusted to 1500 to allow for comparison and subsequent analysis (Lockhart et al., 1996; Wodicka et al., 1997). Analysis was performed.

P53 Mediates Apoptosis Through Both the Mitochondrial and Death Receptor Pathways

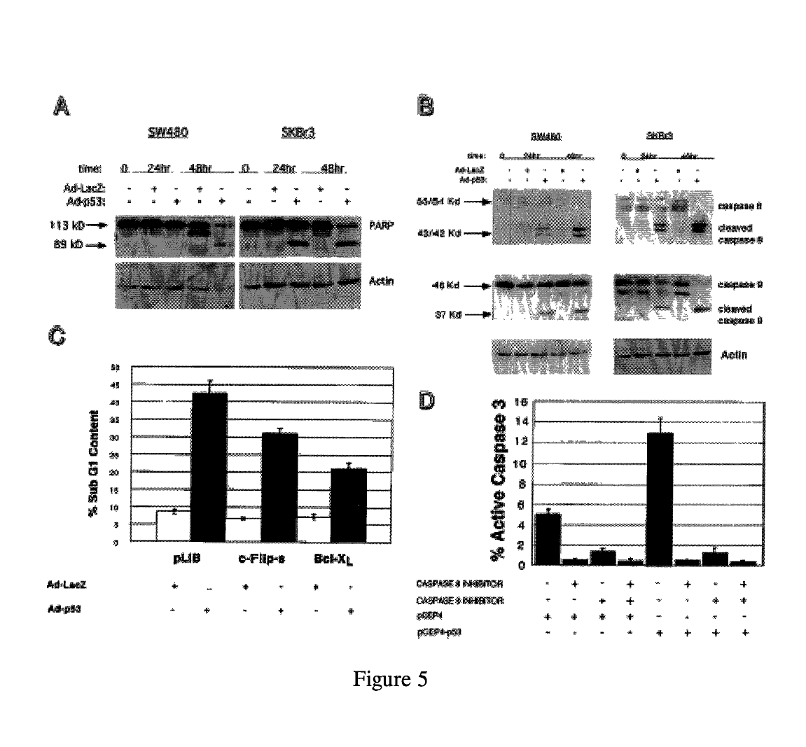

In order to examine the downstream caspases through which p53 induces cell death, this study has infected human cancer cell lines lacking wild-type p53 with an adenovirus expressing wild-type p53 or a control adenovirus containing the LacZ gene. In both the colon carcinoma cell line, SW480, and the breast carcinoma cell line, SKBR3, overexpression of p53 resulted in apoptosis by 48 hours as evidenced by cleavage of the caspase 3 substrate, PARP (FIG. 5a). To elucidate the downstream caspases which are cleaved during p53-mediated apoptosis, this study has examined the cleavage of caspase 8 and caspase 9 after Ad-p53 infection. Western blot analysis revealed that both caspase 8 and caspase 9 were cleaved during p53-dependent apoptosis (FIG. 5b). Furthermore, the cleavage of caspase 8 appeared to precede the cleavage of caspase 9, suggesting that activation of caspase 8 may be independent of caspase 9 activation.

To determine the relative contributions of caspase 8 and caspase 9 to p53-induced cell death, this study has over-expressed p53 in SW480 cell lines that stably over-expressed either c-FLIP-s or Bcl-XL which are specific inhibitors of caspase 8 and caspase 9 activation respectively (Irmler et al., 1997; Kluck et al., 1997; Zou et al., 1997; Tschopp et al., 1998; Adams and Cory, 1998). Either overexpression of c-Flip-s or Bcl-XL was able to partially inhibit p53-mediated death after Ad-p53 infection (FIG. 5c). Furthermore this study has examined the ability of specific irreversible inhibitors of caspase 8 (Z-IETD-FMK) and caspase 9 (Z-LETD-FMK) to inhibit p53-dependent apoptosis (FIG. 5d). Overexpression of p53 results in apoptosis in the adrenal carcinoma cell line, SW13, which could be inhibited by either a caspase 8 or caspase 9 inhibitor (FIG. 5d). These results further support a role for the death receptor and mitochondrial pathways in mediating p53-dependent apoptosis.

Similar Levels of Ionizing Radiation-induced p53-dependent Apoptosis in Vivo

To explore the role of p53 targets genes in activating the caspase cascade and inducing cell death, this study has used an in vivo system of p53-mediated apoptosis. The study of p53-mediated apoptosis in vivo allows one to examine the effects of endogenous p53 on its target genes when p53 is expressed at a physiological level.

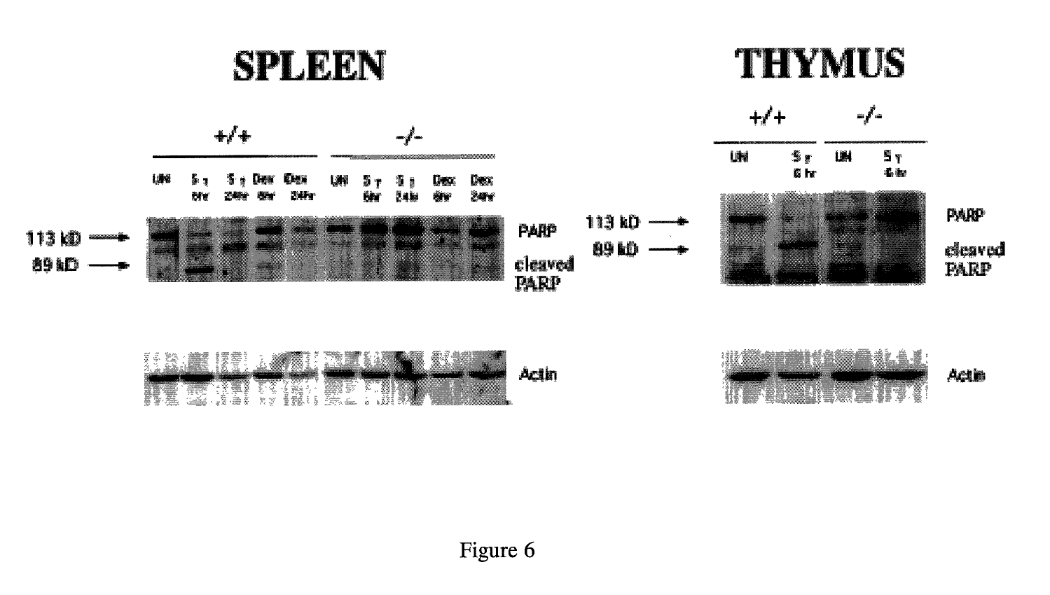

It was decided to determine the relative levels of apoptosis in the thymus and spleen, which have previously been shown to undergo a p53-dependent apoptosis in response to ionizing radiation (Lowe et al., 1993, Midgley et al., 1995). p53+/+ and age matched p53−/− mice were treated with 5 Gy of ionizing radiation or dexamethasone and harvested tissues at 0, 6, and 24 hours after treatment. This study has confirmed that ionizing radiation results in a p53-dependent apoptosis in the thymus and spleen as PARP cleavage was only observed in tissues from p53+/+ animals (FIG. 6). Furthermore this study has observed similar levels of apoptosis as measured by PARP cleavage in thymus and spleen (FIG. 6). Because similar levels of apoptosis were present in these tissues, this study has determined whether the gene expression pattern of known p53 target genes involved in apoptosis were similar after treatment with ionizing radiation in vivo.

p53 Target Genes Involved in Apoptosis are Expressed in a p53-dependent and Tissue-specific Manner Following Exposure to γ Irradiation in Vivo

The mRNA expression level of five p53 target genes in three tissues which undergo p53-dependent apoptosis were analyzed. This study analyzed p21WAF1, bax, Fas/APO1, KILLER/DR5 and E124/PIG8. p21WAF1 is a known cyclin-dependent kinase inhibitor involved in p53-dependent growth arrest while the other four targets have been implicated in p53-dependent apoptosis (El-Deiry et al., 1993; Zhao et al., 2000). Bax and EI24/PIG8 have previously been shown to mediate cell death through the caspase 9 and the mitochondrial pathway while Fas/AP01 and KILLER/DR5 induce death through caspase 8 (reviewed in Burns and El-Deiry, 1999). To determine the mRNA expression levels of the five targets, this study has developed a real-time quantitative RT-PCR assay with expression levels of p53 target genes normalized to endogenous GAPDH mRNA levels. The result of four independent trials revealed that in the spleen, thymus and small intestine, there was no statistically significant difference in the basal expression level of the five p53-target genes in p53+/+ and p53−/− animals (data not shown). The expression of these five p53-target genes was examined after treatment with dexamethasone or 5 Gy of ionizing radiation in p53 wild-type and p53 null animals.

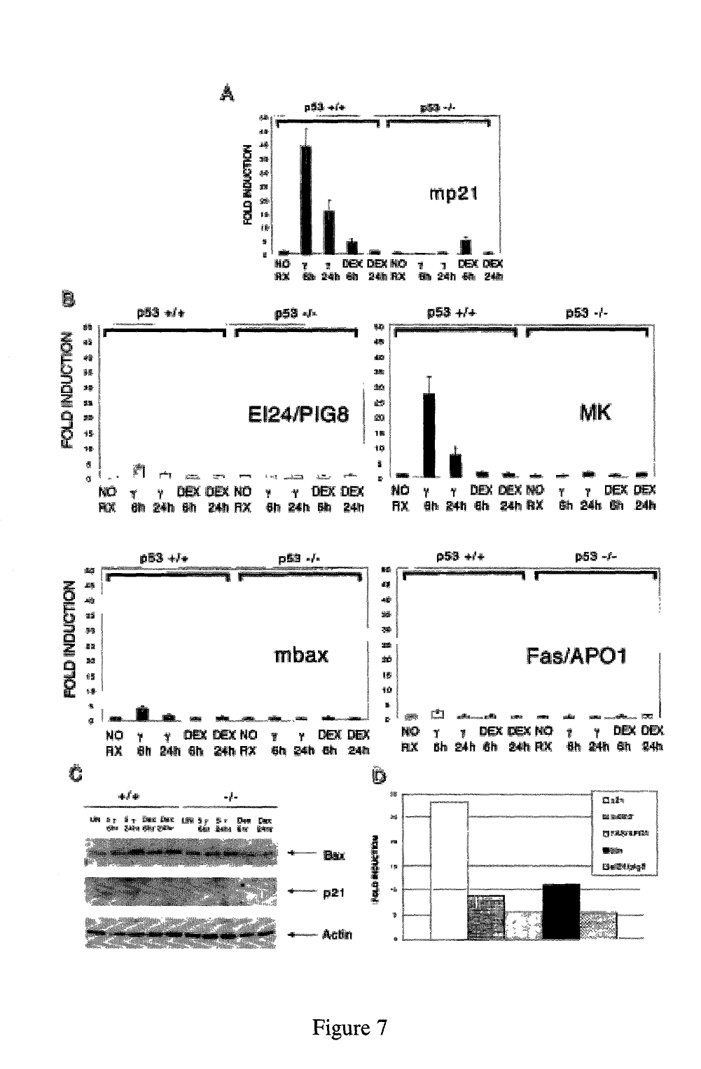

Previous studies and the data above (FIG. 6) have shown that the spleen undergoes p53-dependent apoptosis after γ-irradiation (Midgley et al., 1995). The expression of p21WAF1 was examined and as expected was strongly induced after 5 Gy in the p53+/+ animals by 6 hours and was still elevated 24 hours after treatment but was unchanged in p53−/− animals (FIG. 7a). Interestingly, p21WAF1 was also induced over fourfold by dexamethasone treatment at 6 hours in a p53 independent manner (FIG. 7a). However, the dexamethasone treatment did not cause cell death in the spleen (FIG. 6). The expression levels of bax, Fas/APO1, KILLER/DR5 and EI24/PIG8 were examined. Although these four apoptosis-inducing genes were upregulated in a p53-dependent manner after γ-irradiation, they differed greatly in their extent of induction. While the mRNA levels of bax and EI24 were elevated approximately fourfold at 6 hours, the mRNA level of mouse KILLER/DR5 (MK) was increased almost 30-fold at 6 hours after γ-irradiation. The p53-dependent induction after irradiation of mouse KILLER/DR5 agrees with previous published reports that the KILLER/DR5 promoter contains p53 binding sites and that KILLER/DR5 is induced in a p53-dependent manner after DNA damage in human and mouse tumor cell lines (Wu et al., 1997, 1999; Takimoto and El-Deiry, 2000). In contrast, Fas/APO1 was only weakly induced (2.4-fold) by γ-irradiation in the spleen (FIG. 7b). Western blot analysis for p21 and bax confirmed that these targets were induced in a p53-dependent manner (FIG. 7c). Furthermore microarray analysis confirmed that p21, bax, FAS/APO1, EI24/PIG8 as well as mdm2 exhibited a similar pattern of induction relative to each other as observed by RT-PCR assay (FIG. 7d, data not shown). It is expected that KILLER/DR5 plays a more significant role in mediating p53-dependent apoptosis in the spleen as compared to other targets such as bax or FAS/APO1.

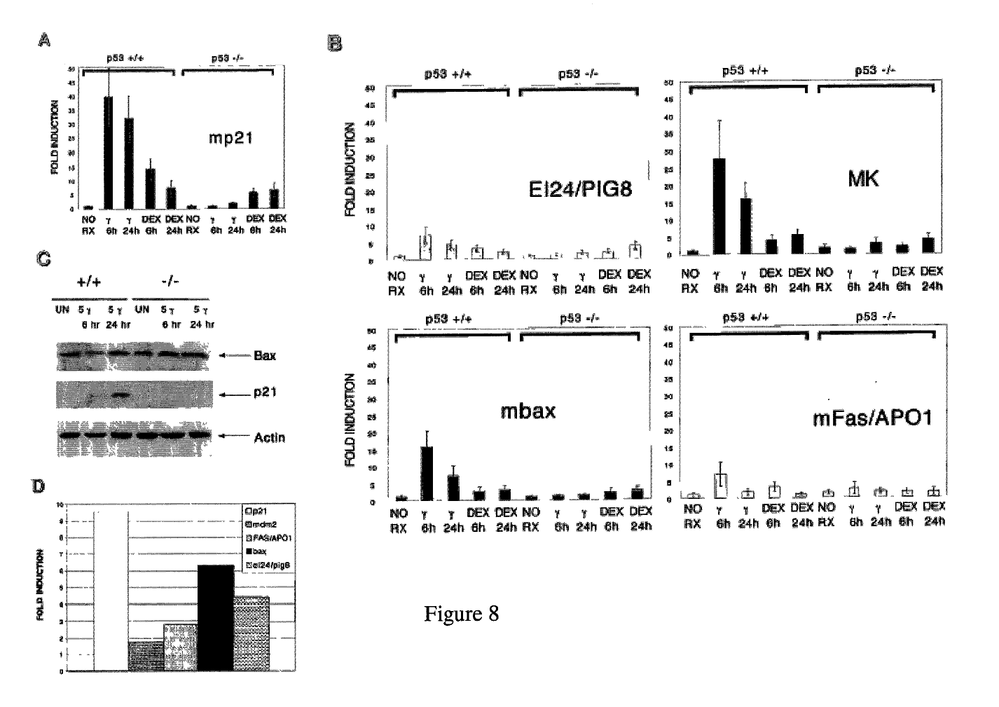

The expression pattern of p21WAF1, bax, Fas/APO1, KILLER/DR5 and EI24/PIG8 was analyzed in the thymus after γ-irradiation. The mRNA expression of p21WAF1 was dramatically increased by 6 hours and remained elevated at 24 hours after γ-irradiation in a p53-dependent manner (FIG. 8a). As observed in the spleen, p21 mRNA was also induced by dexamethasone treated in a p53-independent manner (FIG. 8a). Several studies have demonstrated that dexamethasone treatment results in a p53-independent apoptosis (Lowe et al., 1993). Interestingly, KILLER/DR5 was also induced by dexamethasone at 24 hours after treatment in a p53-independent manner (FIG. 8b). This agrees with previous studies in human cancer cell lines where it was observed that dexamethasone induced apoptosis correlated with KILLER/DR5 induction regardless of p53 status (Meng and El-Deiry, 2001). As observed in the spleen, all five p53 target genes were induced in the thymus by ionizing radiation in a p53-dependent manner. However, unlike the spleen, all four of the apoptotic targets examined were increased by at least sevenfold at 6 hours (FIG. 8b). KILLER/DR5 was again induced almost 30-fold at 6 hours after irradiation and remained elevated 24 hours after treatment. Bax mRNA was also significantly increased (15.7-fold) and remained elevated at 24 h. Microarray analysis confirmed these findings as p21, bax, FAS/APO1, EI24/PIG8 as well as mdm2 exhibited a similar pattern of induction relative to each other as observed by RT-PCR assay (FIG. 8d, data not shown). It is possible that some post-transcriptional mechanism may further regulate the levels of Bax protein. Western blot analysis for p21 and bax confirmed that the target proteins were induced in a p53-dependent manner, however, Bax protein did not seem to be as significantly induced compared to the induction observed at the mRNA level (FIG. 8c). These results suggest that several target genes may be making a significant contribution to the p53 induced cell death in the thymus.

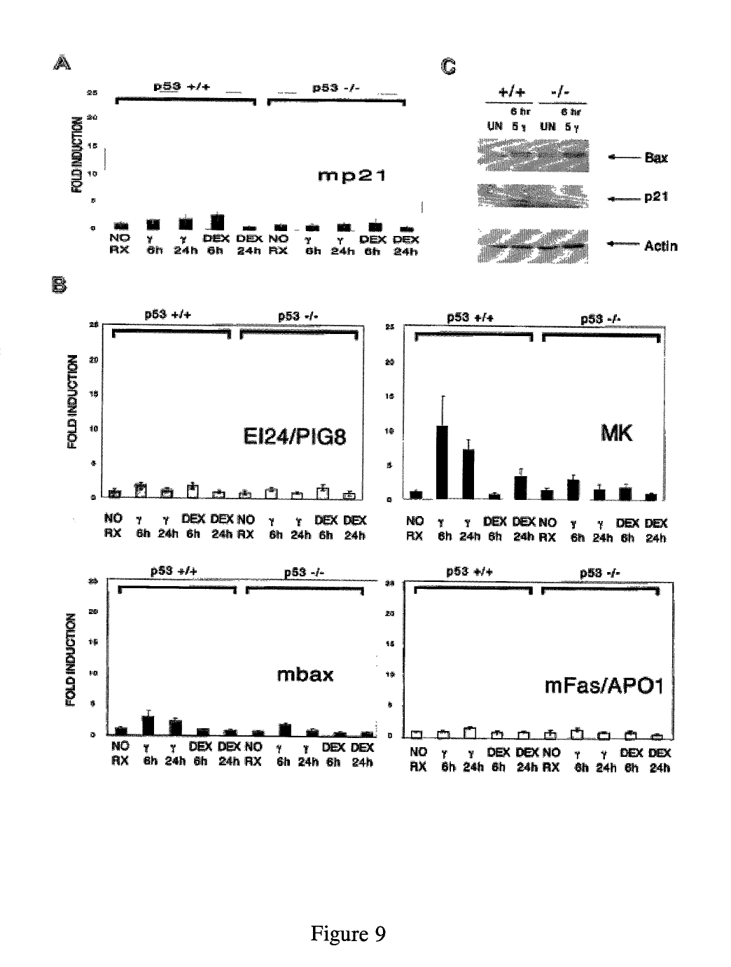

Finally this study has examined small intestine in which cells of epithelial origin have been shown to undergo p53-dependent apoptosis after exposure to γ-radiation (Pritchard et al., 1999). Although p53-dependent apoptosis was observed in the small intestine after ionizing radiation, KILLER/DR5 was the only p53 target gene whose mRNA levels significantly increased after γ-irradiation (FIG. 9a, b). KILLER/DR5 mRNA was induced 10-fold at 6 hours after irradiation and remained elevated at 24 h. Western blot analysis for p21 and bax confirmed these results as p21 was only weakly induced and Bax protein levels did not significantly change (FIG. 9c). These results demonstrate a unique role for KILLER/DR5 in mediating p53-dependent apoptosis in the small intestine following exposure to γ-radiation.

Discussion

To examine the mediators of p53-dependent apoptosis, this study has used both tissue culture and in vivo models of p53-dependent cell death. Previous studies have shown an important and in some cases essential role for the mitochondria and caspase 9 in p53 induced apoptosis (Hakem et al., 1998; Soengas et al., 1999). The role of caspase 8 in p53-dependent death is not well understood. Two target genes of p53 studied here, KILLER/DR5 and FAS/APO1, signal through cleavage of caspase 8 and when overexpressed lead to massive apoptosis (Wu et al., 1997, Owen-Schaub et al., 1995). In this study, it has been shown that caspase 8 is cleaved at the same time or earlier than caspase 9 after p53 overexpression, indicating that p53 is not activating caspase 8 through caspase 9 and 3 indirectly. Furthermore, both caspase 8 and caspase 9 appear to contribute to the observed p53-mediated cell death as either inhibitors of caspase 8 and caspase 9 activity respectively blocked p53-mediated apoptosis. Since this model of p53-dependent apoptosis depended on overexpression of p53 above physiological levels, this study next examined a model of endogenous p53-dependent apoptosis in vivo.

Based on in vivo studies, this study has determined that p53-dependent apoptosis occurs through different mediators depending on the tissue type. Further, based on these findings, it is expected that in some cases apoptosis mediated through p53 may occur by redundant pathways or by a “group effect,” while in other tissues one or few targets may play a key role in p53-dependent apoptosis. There is some support for a single target gene being essential, as studies in the CNS revealed a key role for bax in mediating p53-dependent apoptosis (Chong et al., 2000). The tissue type differences observed in the expression of p53 target genes cannot be explained by differences in the amount of apoptosis in each tissue as this study has observed similar levels of death after irradiation in the spleen and thymus. In the thymus this study has found that bax, EI24/PIG8, FAS/APO1 and KILLER/DR5 in irradiated thymus are significantly induced, suggesting that p53 mediates its cell death through several redundant targets. Because p53 induces all of these genes strongly in the thymus one would predict that the loss of a single target would not attenuate p53-dependent apoptosis. Previous studies agree with this prediction as either bax or FAS/APO1 but not p53 were dispensable in the thymus for DNA damage-induced cell death (Knudson et al., 1995; Fuchs et al., 1997) However, it is still possible that a single target gene may be essential for apoptosis, as knockout animals of many p53 target genes including KILLER/DR5 and EI24/PIG8 have not yet been tested in γ-radiation experiments.

In contrast to the thymus, the spleen and small intestine exhibit a more restrictive expression pattern of p53 target genes during apoptosis. In spleen, Fas/APO, EI24/PIG8 and bax were either induced weakly or not at all. KILLER/DR5 was strongly induced and was the dominant induced apoptotic p53 target gene in these tissues. In the small intestine, an example of p53-dependent death in epithelial tissue, KILLER/DR5 was the only p53 target gene examined in this study which was induced. Based on these results, it is expected that in these two tissues, one or few p53 target genes are essential for p53-dependent death. There are no published reports that either FAS/APO1 or bax is required for apoptosis after ionizing radiation in the spleen. However, the weak induction of these target genes and EI24 suggests that they probably are not playing a key role in p53-dependent apoptosis in these tissues. Previous studies in the small intestine have shown that bax is not required for p53-dependent apoptosis (Pritchard et al., 1999). This result agrees with the findings that bax is not significantly induced in the small intestine. This evidence demonstrates a key role for KILLER/DR5 in mediating p53-dependent apoptosis in the spleen and especially the small intestine. Additional evidence regarding whether KILLER/DR5 is an essential gene for mediating p53-dependent death or one of a few critical targets will be determined by examining the KILLER/DR5 knockout animals for defects in radiation induced apoptosis. It is expected that the requirement for one or multiple target genes in a particular tissue may be dependent upon the balance of pro- or anti-apoptotic molecules in that particular tissue.

Although definitive evidence for an essential role for most p53 targets in vivo is lacking, several animal studies have shown a critical role for caspase 9 is mediating DNA damage induced apoptosis in both the thymus and spleen (Hakem et al., 1998). Based on this data, it is expected that in these tissues KILLER/DR5 plays an important role in mediating p53-dependent apoptosis. Since caspase 9 but not bax has been shown to be required for radiation-induced death in both the thymus and spleen, it is expected that the explanation for the dissociation between a requirement for bax versus caspase 9 is that in these tissues the cleavage of Bid downstream of caspase 8 activation by KILLER/DR5 leads to the eventual activation of caspase 9. Several studies have shown that both Fas and TRAIL treatment can lead to Bid cleavage and activation of caspase 9 (Li et al., 1998; Yamada et al., 1999). Furthermore, there is at least one in vivo example in which Fas requires the mitochondrial pathway for inducing apoptosis (Yin et al., 1999).

The requirement of KILLER/DR5 and caspase 8 in mediating p53-dependent apoptosis in the thymus and spleen can be tested by irradiating the Bid−/− or caspase 8−/− animals and observing whether a cell death defects exists in the thymus or spleen after irradiation. However, even if Bid−/− tissues are not deficient in γ-radiation-induced apoptosis, there may be redundant or yet to be discovered connections between the cell membrane and mitochondrial death pathways. In the small intestine, there is no published evidence that caspase 9 is required for p53-dependent apoptosis and bcl-2 knockout animals have only a small increase in apoptosis after irradiation, suggesting that the mitochondrial pathway may not be as important in the small intestine (Pritchard et al., 1999). Interestingly, KILLER/DR5 was the only p53 target gene examined that was significantly upregulated in the small intestine following exposure to γ-radiation. Based on these results, it is expected that KILLER/DR5 plays a unique role in mediating p53-dependent apoptosis in the small intestine. It is expected that irradiation studies of the KILLER/DR5 and Bid knockout animals and examination of the resulting apoptosis in the small intestine would in the future address remaining questions.

Given the expected unique role of KILLER/DR5 in mediating p53-dependent death after ionizing radiation in vivo, it is not surprising that mutations of KILLER/DR5 in head and neck and cancer, and in non-small cell lung cancers have been found (Pai et al., 1998; Lee et al., 1999). Further support for a key role for KILLER/DR5 in human cancer comes from previous reports which demonstrated that the anti-apoptotic TRAIL decoy receptor, TRID/TRAIL-R3 is overexpressed in primary tumors of the gastrointestinal tract (Sheikh et al., 1999). Because KILLER/DR5 is uniquely induced by p53 in the small intestine after γ-irradiation, it may be necessary for primary tumors of the gastrointestinal tract to upregulate TRID/TRAIL-R3 to prevent p53-dependent apoptosis (Sheikh et al., 1999; Meng et al., 2000). It is expected that loss of KILLER/DR5 may substitute for loss of p53 or at least may increase a tumor cell's tolerance to wild-type p53.

In summary, this study has demonstrated in two human tumor cell lines that both caspase 8 and caspase 9 are cleaved during p53-dependent apoptosis and that inhibitors of death receptor or mitochondrial death signaling inhibit p53-mediated cell death. These studies in vivo have revealed striking tissue specific patterns of expression for p53 target genes. This data demonstrates that p53-mediated apoptosis occurs in some tissues such as the thymus through redundant pathways or by a “group effect,” while in other tissues one or few targets may play a key role in p53-dependent apoptosis. Finally this study has found evidence that KILLER/DR5 is playing a key role in mediating p53-dependent apoptosis in vivo and has a unique role among transcriptional targets of p53, causing cell death in response to γ-irradiation in the small intestine.

Detailed Description of FIGS. 5-9

FIG. 5. Infection of colon carcinoma cell line SW480 and breast carcinoma cell line SKBr3 with Ad-p53 results in caspase cleavage and apoptosis. (a) Western blot analysis for PARP cleavage after adenovirus infection. SW480 and SKBr3 cell lines were infected with an MOI of 50 with either Ad-LacZ or Ad-p53 and harvested at 0, 24 and 48 hours after infection. Actin was used to confirm that an equivalent amount of protein was loaded in each lane. (b) Western blot analysis of caspase 8 and 9 cleavage after adenovirus infection. (c) FACS analysis for percentage of cells with a sub-G1 content after adenoviral infection with Ad-LacZ or Ad-p53 of SW480 stably overexpressing pLIB (control vector), pLIB-c-Flip-s or pLIB-Bcl-XL. 2.5×10⁵ cells were infected at an MOI of 50 with either Ad-LacZ or Ad-p53 and cells were harvested at 48 hours after infection for FACS analysis. The Sub-G1 content of 20,000 cells was examined for each sample. This experiment was performed in triplicate. (d) FACS analysis for active caspase 3 in adrenal carcinoma cell line SW13 after transfection with p53 in the presence or absence of irreversible caspase 8 inhibitor or irreversible caspase 9 inhibitor. 5.0×10⁶ cells were transfected with 1.8 pg of pCEP4-p53 or control vector and 0.200 pg of pEGFP-Spectrin. Four hours after transfection either the irreversible caspase 8 inhibitor, Z-IETD-FMK or irreversible caspase 9 inhibitor, Z-LEHD-FMK or DMSO was added to the media at a final concentration of 20 μM. Cells were harvested at 24 hours. The percentage of cells expressing activated caspase 3 for 10 000 GFP positive cells were examined for each sample. This experiment was performed in triplicate. The chart depicts percentage of transfected cells which express activated caspase 3.

FIG. 6. Ionizing radiation induces a p53-dependent apoptosis in the spleen and thymus. Western analysis of PARP cleavage after treatment in p53+/+ and p53−/− animals. Two animals per condition received 0.5 mg dexamethasone or 5 Gy total body irradiation and tissues were harvested at 0, 6, and 24 hours after treatment. Left panel: spleen right panel: Thymus. Westerns are from individual (unpooled) tissues at each condition. Actin was used to confirm that an equivalent amount of protein was loaded in each lane.

FIG. 7. Expression of p53 target genes in the spleen after irradiation or dexamethasone treatment in p53+/+ and p53−/− animals. (a) p21 mRNA expression in p53+/+ animals and p53−/− animals after irradiation or dexamethasone treatment. (b) mRNA levels of four p53 target genes (upper left: EI24/PIG8; upper right: mouse KILLER/DR5 (MK); lower left: bax; lower right: Fas/APO1). mRNA expression levels were determined by real time quantitative RT-PCR assay (see Materials and methods). Results from individual tissues are shown. All expression levels are normalized to GAPDH in each well. Fold induction is defined as the fold increase for each sample relative to p53+/+ untreated animals. (c) Western blot analysis for bax and p21 after irradiation or dexamethasone treatment. Actin was used to confirm that an equivalent amount of protein was loaded in each lane. (d) Oligonucleotide microarray analysis of total RNA from the spleen of irradiated and untreated p53+/+ and p53−/− animals for p21, mdm2, FAS/APO1, bax, and ei24/pig8. The chart depicts the fold induction of p21, mdm2, FAS/APO1, bax, and ei24/pig8 6 hours after 5 Gy irradiation relative to the wild type untreated animals.

FIG. 8. Expression of p53 target genes in the thymus after irradiation or dexamethasone treatment in p53+/+ and p53−/− animals. (a) p21 mRNA expression in p53+/+ animals and p53−/−animals after irradiation or dexamethasone treatment. (b) mRNA levels of four p53 target genes (upper left: EI24/PIG8; upper right; mouse KILLER/DR5 (MK); lower left: bax; lower right: Fas/APO1). mRNA expression levels were determined by real time quantitative RT-PCR assay (see Materials and methods). Results from individual tissues are shown. All expression levels are normalized to GAPDH in each well. Fold induction is defined as the fold increase for each sample relative to p53+/+ untreated animals. (c) Western blot analysis for bax and p21 after irradiation. Actin was used to confirm that an equivalent amount of protein was loaded in each lane. (d) Oligonucleotide microarray analysis of total RNA from the thymus of irradiated and untreated p53+/+ and p53−/− animals for p21, mdm2, FAS/APO1, bax, and ei24/pig8. The chart depicts the fold induction of p21, mdm2, FAS/APO1, bax, and ei24/pig8 6 hours after 5 Gy irradiation relative to the wild type untreated animals.

FIG. 9. Expression of p53 target genes in the small intestine after irradiation or dexamethasone treatment in p53+/+ and p53−/− animals. (a) p21 mRNA expression in p53+/+ animals and p53−/− animals after irradiation or dexamethasone treatment. (b) mRNA levels of four p53 target genes (upper left: EI24/PIG8; upper right: mouse KILLER/DR5 (MK); lower left: bax, lower right: Fas/APO1). mRNA expression levels were determined by real time quantitative RT-PCR assay (see Materials and methods). Results from individual tissues are shown. All expression levels are normalized to GAPDH in each well. Fold induction is defined as the fold increase for each sample relative to p53+/+ untreated animals. (c) Western blot analysis for bax and p21 after irradiation. Actin was used to confirm that an equivalent amount of protein was loaded in each lane.

Applicants note that additional experimental details, such as identification of the source/manufacturer of materials and reagents, and primer and probe sequences, is found in U.S. patent application No. 60/232,556 and Applicants' publication, Timothy F. Burns, Eric J. Bernhard, and Wafik S. El-Deiry, Tissue specific expression of p53 target genes suggests a key role for KILLER/DR5 in p53-dependent apoptosis in vivo, Oncogene, 20:4601-4612 (2001), the contents of which are incorporated by reference herein in full.

### Example 2

### Death Domain Mutagenesis of KILLER/DR5 Reveals Residues Critical for Apoptotic Signaling

The Fas/tumor necrosis factor (TNF)/TRAIL receptors signal death through a cytoplasmic death domain (“DD”) containing six a-helices with positively charged helix 2 interacting with negatively charged helix 3 of another DD. DD mutation occurs in head/neck and lung cancer (TRAIL receptor KILLER/DR5) and in lpr mice (Fas). The apoptotic potential of known KILLER/DR5 lung tumor-derived mutants (n=6) and DD mutants (n=18) generated based on conservation with DR4, Fas, Fas-associated death domain (FADD), and tumor necrosis factor receptor 1 (TNFR1) was examined. With the exception of Arg-330 required in Fas or FADD for aggregation or for TNFR1 cytotoxicity, surprisingly major loss-of-function KILLER/DR5 alleles (W325A, L334A (lpr-like), I339A, and W360A) contained hydrophobic residues. Loss-of-function of I339A (highly conserved) has not been reported in DDs. Charged residue mutagenesis revealed that K331A, D336A, E338A, K340A, K343A, and D351A have partial loss-of-function suggesting multiple charges stabilize receptor-adapter interactions. Analysis of the tumor-derived KILLER/DR5 mutants revealed the following: 1) L334F has partial loss-of-function versus L334A, whereas E338K has major loss-of-function versus E338A, examples where alanine and tumor-specific substitutions have divergent phenotypes; 2) unexpectedly, S324F, E326K, K386N, and D407Y have no loss-of-function with tumor-specific or alanine substitutions. Loss-of-function KILLER/DR5 mutants were deficient in recruitment of FADD and caspase 8 to TRAIL death-inducing signaling complexes. The results reveal determinants within KILLER/DR5 for death signaling and drug design.

Materials and Methods

Alanine Scanning Mutagenesis of KILLER/DR5-The KILLER/DR5 cDNA was cloned in frame into pcDNA3.1-Myc-HisA as an EcoRI/HindIII fragment. This C-terminally tagged Myc-His plasmid was subsequently mutagenized using site-directed mutagenesis. Due to the size of the template (6.7 kilobases), certain changes were made to the standard protocol to yield polymerase chain reaction product: inclusion of 10% glycerol, 5% Me₂SO, and 100 ng of template in polymerase chain reaction, decrease of annealing temperature to 50° C., and 18 cycles of amplification. Mutations were verified by sequencing, and in each case the entire cDNA was checked for the absence of second site mutations.

Transfections, in Vitro Translation, and Western Blotting. The colon cancer cell line SW480 was maintained and transfected as described previously (Wu, G. S., et al., Nat. Genet. 17:141-143 (1997)). Protein extracts were harvested in 1×Laemmli sample buffer 16 hours after transfection. PARP and Myc immunoblots were performed following SDS-polyacrylamide gel electrophoresis. Horseradish peroxidase-conjugated secondary antibody (1:5000) treatments were followed by enhanced chemiluminescence. In vitro translation reactions were carried out using 1 μg of wild-type or mutant KILLER/DR5 plasmid DNA and a T7-coupled reticulocyte lysate system.

GFP/PI Fluorescence-activated Cell Sorting Analysis for Sub-G1 Peak. SW480 cells were transfected with a 1:10 ratio of EGFP-spectrin (Kalejta, R. F., et al., Mol. Ther. 1:130-144 (2000)) and KILLER/DR5 expression plasmid. Transfections were harvested at 18 hours after transfection, processed, and analyzed for the presence of a sub-G1 peak as described previously (Ozoren, N., et al., Int. J. Oncol. 16:917-925 (2000)). 10,000 GFP-positive cells were analyzed per experiment, and three independent experiments were performed for each mutant. Percentage of apoptosis was calculated by subtracting transfection-induced sub-G₁ peak (vector transfection) from each wild-type or mutant KILLER/DR5 sub-G₁ peak. Figures for Tables II and III were generated by setting wild-type death to 100% (˜40% sub-G₁ peak at 18 hours) in order to compare point mutants to the wild-type protein.

Blue Cell Method. The blue cell method was performed as previously described (Meng, R. D., et al., Mol. Ther. 1:130-144 (2000)). Calculations for Table I were carried out by setting the number of vector-transfected blue cells to 100% (at least 300 cells for each independent experiment), and the wild-type and point mutants were determined as a percentage of vector transfected.

TRAIL DISC Immunoprecipitation. 293 HEK cells were plated to achieve 80% confluence at the time of transfection in a T75. 30 ug of the indicated KILLER/DR5 plasmid was transfected by calcium phosphate for 16 hours to minimize loss of transfected cells. The cells were then trypsinized, spun down, and resuspended in 2 ml of complete medium supplemented with 50 ng/ml His tagged-TRAIL and 1 μg/ml anti-6-histidine antibody for 15 minutes at 37° C. For untreated samples, only the TRAIL was excluded. The cells were washed twice with ice-cold phosphate-buffered saline and lysed for 30 minutes on ice in TRAIL DISC IP lysis buffer (30 mM Tris, pH 7.5, 150 mM NaCl, 10% glycerol, 1% Triton X-100). The lysates were cleared twice by centrifugation at 4° C. The supernatants were immunoprecipitated overnight with 30 μl of agarose at 4° C. to isolate the TRAIL DISC. The complexes were subsequently washed four times with TRAIL DISC IP lysis buffer and eluted with elution buffer with 0.1 M dithiothreitol at room temperature for 2 hours. The protein complexes were methanol/chloroform-precipitated and resolved on 15% SDS-polyacrylamide gels. Caspase 8 (1:1000) and FADD (1:2000) Western blots were performed to measure recruitment of these endogenous proteins to the TRAIL DISC along with the exogenously expressed KILLER/DR5 (Myc) protein.

Results

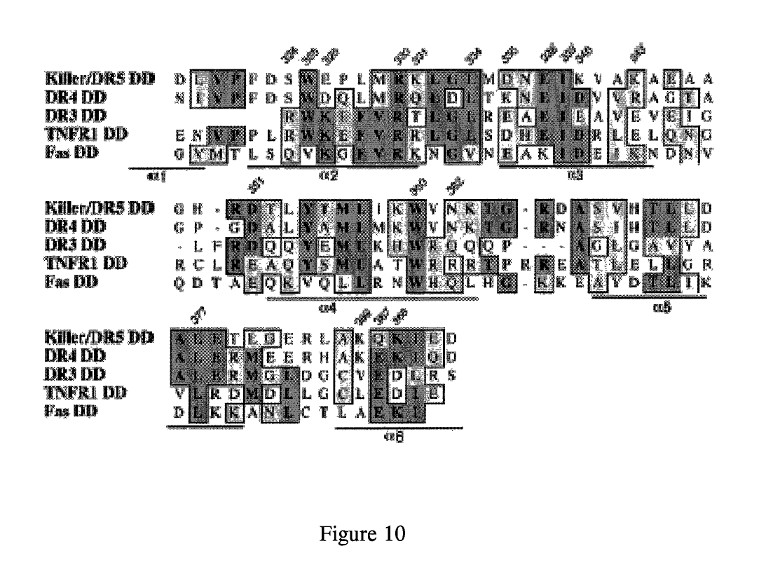

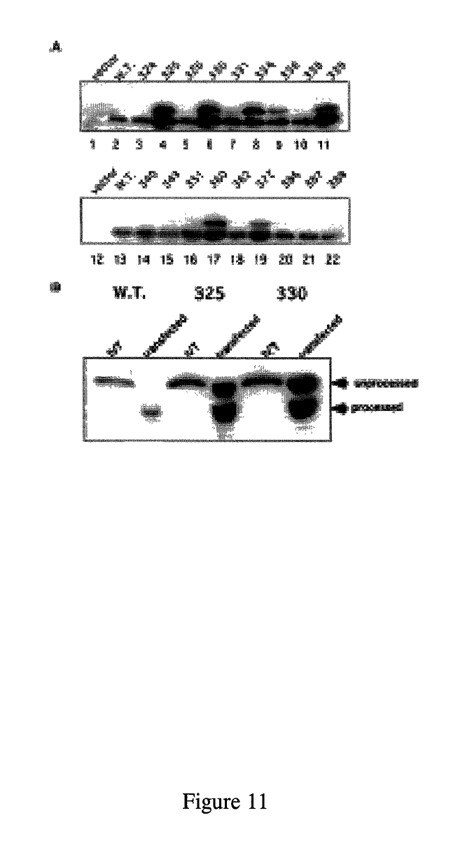

Design of KILLER/DR5 DD Alanine Scanning Mutants Based on Homology to DDs of the TNFR Superfamily. Recent mutational and crystal structure studies with Fas and FADD have revealed the importance of helices 2 and 3 of both proteins. Electrostatic interactions between surface residues are thought to mediate DD/DD interactions. DD mutants were designed, based on a number of selection criteria including residue charge, hydrophobicity, conservation, and demonstrated functional significance in other receptor systems. Due to the noted importance of helices 2 and 3 within Fas and FADD, this study focused on the mutagenesis of charged residues in order to identify residues crucial for receptor/adapter interactions. FIG. 10 depicts an amino acid alignment of the death domains of selected members of the TNF superfamily. Eighteen residues within KILLER/DR5, which were targeted for alanine replacement mutagenesis, are noted with an asterisk along with the amino acid position of the short form of the KILLER/DR5 protein. FIG. 11A shows protein expression of each construct following transfection into SW480 colon cancer cells for 16 hours. Most of the alanine mutants expressed protein at levels equivalent to wild type KILLER/DR5. Selected mutants showed an increase in protein expression levels in addition to the appearance of a lower mobility form of the protein. Interestingly, these mutants with higher levels of expression and the larger form appeared to be either partially or completely defective in inducing apoptosis (see data below). This would explain the higher levels of the transfected protein as the cell was able to withstand the presence of these non-toxic proteins. The lower mobility form could also be explained due to the higher levels of transfected protein being made and withstood by the cell. FIG. 11B compares transfected cell lysates to in vitro translated proteins, which do not undergo processing of the signal peptide. The TNT reactions yield a lower mobility form of KILLER/DR5, which comigrates with the upper band observed in the transfected cell lysates of mutants, W325A and R330A. In addition, cotransfection of the anti-apoptotic gene, FLIPS, along with wild type KILLER/DR5 protects the cells from death and both the processed and unprocessed forms of the wild-type protein can be detected by Western blot analysis (data not shown).

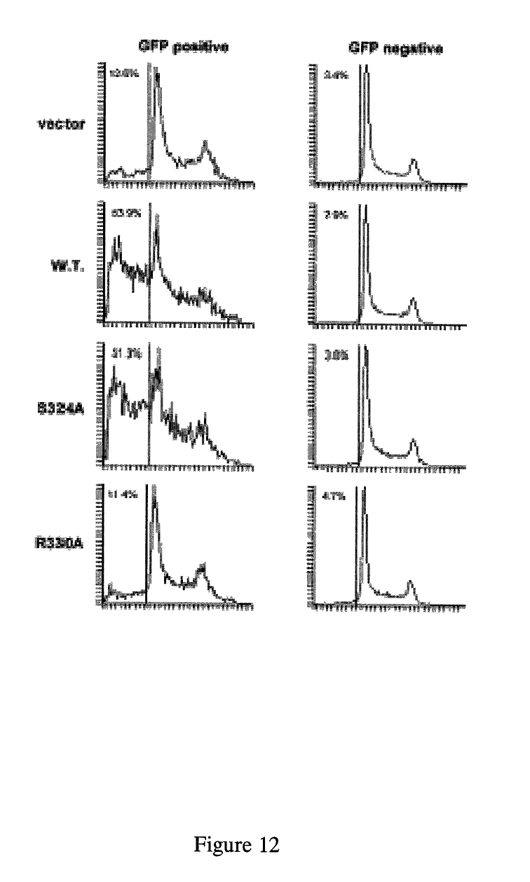

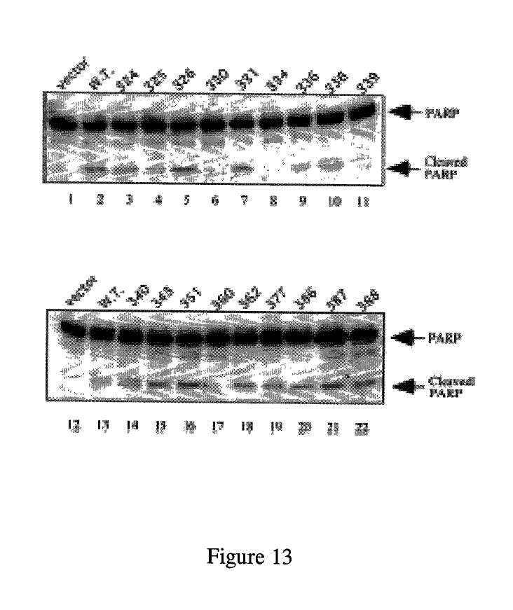

Conserved Hydrophobic Residues Are More Critical for Apoptotic Signaling as Compared with Conserved Charged Positions in the KILLER/DR5 DD. In order to functionally characterize the mutant receptors, this study employed three methods to assess cell death following transfection. The first method involved cotransfection of the mutant receptor along with pCMVb gal. Blue cells were counted after 40 hours in order to determine relative cell survival as compared with vector-transfected cells (number of blue cells for vector was set as equivalent to 100% survival; Table I). To specifically measure apoptosis, this study used a cotransfection based flow cytometric assay to functionally assess apoptotic signaling of each substitution mutant. Each mutant was cotransfected with an EGFP-spectrin construct at a 10:1 ratio (mutant plasmid:EGFP-spectrin plasmid) into SW480 cells for 18 hours and the presence of a sub-G₁ peak was used to quantitate apoptotic signaling. The use of the spectrinbound GFP allowed for the identification of specifically transfected cells, and the spectrin fusion permits cell membrane retention of GFP even if cells lose membrane permeability due to cell death (Kalejta, R. F., et al., Mol. Ther. 1:130-144 (2000)). FIG. 12 shows an example of a typical experiment with GFP-positive cells in the left column and GFP negative cells in the right column. Vector-transfected (GFP(+)) cells displayed minimal toxicity (as compared with the GFP(−) counterparts) associated with the transfection of DNA into the cells. In contrast, the transfection of wild-type KILLER/DR5 dramatically increased the sub-G1 peak in addition to decreasing the G₂ peak as compared with the untransfected cell population (FIG. 12). One example of a signaling-competent and -incompetent mutant is also shown for comparison. Table II lists the results of three independent experiments for each mutant compared with the wild-type protein after subtracting out transfection-induced death. Finally, as a further demonstration of each mutant's ability to induce apoptosis, PARP cleavage was measured following 16 hours of transfection. FIG. 13 illustrates these results with the arrow indicating the cleaved form of the PARP protein, indicative of apoptosis. Vector-transfected cells showed no sign of PARP cleavage at 16 hours, whereas wild-type KILLER/DR5 efficiently caused PARP cleavage (FIG. 13, compare lanes 1 and 2).

The data from the three methods correlated well with one another (Tables I and II and FIG. 13) and yielded three groups into which the alanine mutants could be divided based on phenotype. The first group contained five mutants, which have a dramatically reduced ability to induce apoptosis: W325A, R330A, L334A (lpr-like), I339A, and W360A. The cells expressing these mutants retained ˜75% blue cell staining (as compared with 8% for wild type; Table I), demonstrated at least a 75% reduction in sub-G₁ peak at 18 hours in four out of the five mutants (Table II), and displayed little to no PARP-cleaving activity at 16 hours (FIG. 13). This also correlated with the observation on the protein level that these five mutants had high levels of processed and unprocessed receptor that the cells were able to withstand due to its lack of toxicity (FIG. 11A, lanes 4, 6, 8, 11, and 17). Interestingly, four of these (including the lpr-like mutation) represent hydrophobic residues scattered throughout helices 2, 3, and 4, which when mutated probably affect overall protein structure due to disruption of the hydrophobic core. Disruption of the lpr position in either Fas or FADD results in an inability of the proteins to interact and in TNFR1 a loss of cytotoxicity. Of these five mutants, only R330A might play a role in potentially mediating an electrostatic death domain interaction. This charged residue, which is completely conserved in every proapoptotic TNFR family member, also is known to be critical in TNFR1, Fas, and FADD, thereby highlighting its importance in transducing an apoptotic signal.

TABLE I
Blue cell assay for alanine scanning mutants as a
percentage of vector

| | Blue cells |
|---|---|
| KILLER/DR5 Alanine point mutant | % of vector |
| 3.1 Myc-His vector | 100 |
| Wild-type KILLER/DR5 | 8 ± 0.2 |
| Loss-of-function mutations | |
| W325A | 72 ± 5.0 |
| R330A | 86 ± 2.9 |
| L334A (position equivalent to Fas lpr) | 86 ± 14.4 |
| 1339A | 82 ± 31.3 |
| W360A | 79 ± 3.1 |
| Partial loss-of-function mutations | |
| K331A | 37 ± 15.7 |
| D336A | 50 ± 1.8 |
| E338A | 34 ± 3.2 |
| K340A | 53 ± 4.1 |
| K343A | 47 ± 2.8 |
| D351A | 17 ± 4.4 |
| L377A | 38 ± 2.2 |
| No loss-of-function mutations | |
| S324A | 37 ± 6.4 |
| E326A | 3 ± 0.1 |
| N362A | 10 ± 2.0 |
| K386A | 14 ± 3.6 |
| Q387A | 11 ± 4.3 |
| K388A | 9 ± 1.5 |

TABLE II
Apoptosis (sub-G1) induced by point mutants expressed as
percentage of wild type

| | Sub-G1 peak | |
|---|---|---|
| | Apoptotic ability a | |
| KILLER/DR5 point mutant | % WT | 100 WT |
| Loss-of-function mutations | | |
| W325A | 44 ± 8.4 | — |
| R330A | 21 ± 1.6 | — |
| L334A (position equivalent to Fas lpr) | 28 ± 14.2− | |
| 1339A | 5 ± 3.4 | — |
| W360A | 22 ± 2.5 | — |
| Partial loss-of-function mutations | | |
| K331A | 85 ± 5.2 | ± |
| D336A | 74 ± 11.1 ± | |
| E338A | 83 ± 7.6 | ± |
| K340A | 85 ± 12.4 | ± |
| K343A | 83 ± 8.6 | ± |
| D351A | 90 ± 4.1 | ± |
| L377A | 73 ± 5.0 | ± |
| No loss-of-function mutations | | |
| S324A | 90 ± 12.7 WT | |
| E326A | 96 ± 10.6 WT | |
| N362A | 98 ± 10.9 WT | |
| K386A | 110 ± 7.5 WT | |
| Q387A | 110 ± 9.0 WT | |
| K388A | 101 ± 11.3 | WT |

a WT, able to induce apoptosis as well as wild-type protein;
±, 20-25% reduction in ability to induce apoptosis;
−, completely deficient in ability to induce apoptosis.

The second class of mutants that was identified by this study demonstrated only a partial loss in cell death signaling: K331A, D336A, E338A, K340A, K343A, D351A, and L377A. These mutants, when overexpressed, displayed only a 10-25% reduction in cell death as measured by sub-G1 (Table II) and slightly reduced amount of PARP cleavage as compared with wild-type (FIG. 13). With the exception of L377A, these partially defective mutants represent charged residues within helices 2 and 3 and the boundary of helix 4. Interestingly, previous in vitro binding studies with Fas and FADD revealed a total loss of interaction if a mutation occurred at the positions corresponding to Lys-331, Asp-336, and Lys-340 and a partial loss at Lys-343. Therefore, these partial loss-of-function mutations are expected to indicate residues important in mediating interactions with an adapter molecule.

The final class of mutants are those that are unaffected by alanine substitution: Ser-324, Glu-326, Asn-362, Lys-386, Gln-387, and Lys-388. They function as the wild-type protein does in all three of the aforementioned assays. In the case of position 326, the homologous residue in FADD when mutated and tested shows an inability to interact with Fas in vitro. Examination of charge distribution reveals that, in the case of the TRAIL receptors, DR4 and KILLER/DR5, this residue is negatively charged whereas Fas, FADD, and TNFR1 all retain a positive charge. This residue represents a difference between the TRAIL receptors and the rest of TNFR family DDs not only in charge but also in function. The other residues, which are not affected by mutation, although illustrating examples of subtle differences between family members, are not conserved in identity or charge and to this point have not been demonstrated to be important for function in any of the receptor/adapter systems studied thus far. Meanwhile, mutation of highly conserved hydrophobic residues throughout the DD renders KILLER/DR5 nonfunctional and elimination of charged residues presumed to be the sites of putative protein interactions partially suppresses the apoptotic signal in an overexpression environment.

Some but Not All Tumor-derived KILLER/DR5 Mutants Display Loss of Apoptotic Function When Overexpressed. Due to the incidence of chromosome 8p21-22 loss in human cancer cells and the lack of an identified tumor suppressor gene in the area, groups have turned their attention toward identifying mutations in DR4 and KILLER/DR5, which have both been mapped to 8p2l. The first report of a tumor associated mutation of KILLER/DR5 was in a head neck cancer, which resulted in a truncation of the cytoplasmic domain of the protein. In another study, also looking to assign significance to the KILLER/DR5 gene in chromosome 8p21-22 loss of cancers, Lee et al. reported alterations in the cytoplasmic domain of the protein in non-small cell lung cancers. Out of 104 samples, 11 mutations were reported, including eight missense mutations. This provided us with the opportunity to compare the functional data using alanine mutagenesis to naturally occurring tumor-derived mutants. Three of the tumor mutations occurred at amino acid position 334 (L334F), which corresponds to the same residue altered in the Fas lpr case. Coincidentally, four of the remaining five mutations were targeted in the original alanine mutagenesis of the protein: S324F, E326K, E338K, K386N. The remaining point mutation occurred just four amino acids from the end of the protein, D407Y.

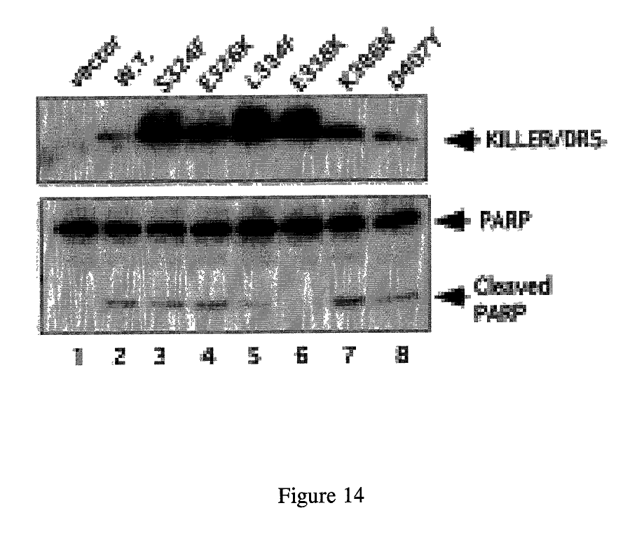

The tumor-derived mutants were generated in the same manner as the alanine mutants in order to compare alanine versus tumor-specific mutant with the wild-type KILLER/DR5 protein. Mutations were verified by DNA sequencing, and protein expression along with PARP cleavage was evaluated (FIG. 14 and data not shown). The most common naturally occurring mutation (L334F; 3 out of 11), which corresponds to the lpr position, actually demonstrated a more severe phenotype as an alanine substitution (Table III). Interestingly, the naturally occurring mutation (L334F) retains the hydrophobicity at the position while decreasing its apoptotic potential by 50%. Conversely, a mutation at a putative protein-protein interaction site (E338K) displayed a complete loss-of-function whereas the alanine mutation displayed only a partial loss-of-function phenotype (Table III). The tumor-specific mutation at position 338 changes the charge from negative to positive, explaining the dramatic loss of apoptotic capacity; however, both Fas and FADD normally have a positively charged lysine at this position. Charge differences such as this between family members provide a clue to receptor/adapter specificity. Nevertheless, the tumor-specific mutant data provides convincing in vivo evidence that positions Leu-334 and Glu-338 are important for proper downstream signaling of cell death.

TABLE III
Apoptosis (sub-G1) induced by tumor point mutant versus
alanine mutant

Sub-G1 peak Apoptotic ability a

| KILLER/DR5 point mutant | % WT | 100 WT |
|---|---|---|
| Loss-of-function mutations | | |
| L334A | 13 ± 9.6 | — |
| L334F | 49 ± 12.8 | —* |
| E338A | 80 ± 12.4 | ± |
| E338K | 8 ± 6.2 | — |
| No loss-of-function mutations | | |
| S324A | 96 ± 8.4 | WT |
| S324F | 80 ± 10.7 WT | |
| E326A | 107 ± 4.3 WT | |
| E326K | 106 ± 5.2 WT | |
| K386A | 105 ± 13.9 | WT |
| K386N | 112 ± 11.0 | WT |
| D407Y | 123 ± 3.2 WT | |

a WT, able to induce apoptosis as well as wild-type protein;
±, 20-25% reduction in ability to induce apoptosis;
−*, 50% reduction in ability to induce apoptosis;
−, completely deficient in ability to induce apoptosis.

Unexpectedly, the remaining four missense mutations (S324F, E326K, K386N, D407Y) displayed no loss of apoptotic signaling as assessed by sub-G₁ (Table III) or by PARP cleavage (FIG. 14) in the overexpression studies. Mutation of these residues in the original alanine-mutagenesis yielded no phenotype as well, strengthening the idea of a lesser role played by these sites in the overall structure and signaling of the molecule. This does not rule out the possibility that in these tumors, many of which may have 8p LOH and hence reduced gene dosage, that this mutation may have subtle effects not detectable in these types of overexpression studies.

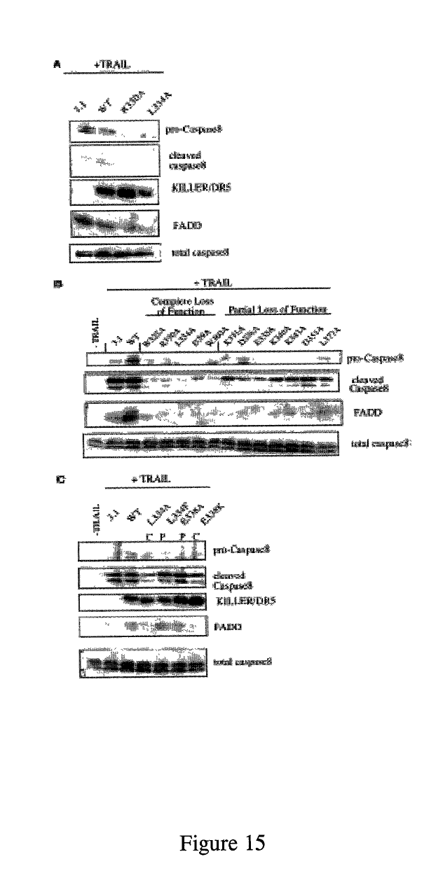

Expression of Partial or Complete Loss-of-function Mutants Diminishes Endogenous FADD and Caspase 8 Recruitment to TRAIL DISCs. In addition to the receptor/ligand trimers, the TRAIL DISC has recently been demonstrated to contain the adapter molecule FADD and caspase 8 in order to propagate the apoptotic signal. Due to an inability to demonstrate a direct interaction between FADD and KILLER/DR5 in vitro, this study attempted to recapitulate this interaction in vivo in the context of the DISC and to determine the effect of exogenous partial and loss-of-function KILLER/DR5 mutants on FADD and caspase 8 recruitment. 293 HEK cells were chosen due to their ability to be transfected and relative resistance to TRAIL-induced apoptosis. Following transfection of the wild type or mutant KILLER/DR5 plasmid, the cells were harvested 16 hours later and treated with His-tagged TRAIL and a crosslinking anti-6-histidine antibody for 15 minutes at 37° C. The DISC was then immunoprecipitated and analyzed for the presence of the exogenous KILLER/DR5 receptor along with FADD and caspase 8. As demonstrated in FIG. 15A, TRAIL treatment led to the recruitment of the exogenous KILLER/DR5 receptor into DISCs as visualized with an anti-myc antibody. Total caspase 8 was provided as a loading control to ensure equivalent amounts of proteins were treated and processed for DISC analysis. FADD and caspase 8 (both pro-and the cleaved p46 form) were detected in the DISC of vector- and wild-type receptor transfected 293 cells; however, introduction of either complete loss-of-function receptor, R330A or L334A, led to a dramatic decrease in both FADD and caspase 8 recruitment. This observation helps to explain the lack of apoptosis induction by these mutants in earlier cell death assays (Tables I and II and FIG. 13).

These observations were extended to the panel of complete and partial loss-of-function alanine mutations (FIG. 15B) as well as the tumor mutants (FIG. 15C). Absence of TRAIL treatment did not result in DISC formation, but TRAIL treatment of vector or wild-type transfected cells showed a robust recruitment of both FADD and caspase 8.

Examination of the amount of cleaved caspase 8 in FIG. 15B clearly correlated with the predicted apoptotic potential of each receptor class. The partial loss-of-function mutants showed a decrease in both cleaved caspase 8 and FADD as compared with wild-type. The complete loss-of-function alanine mutants have an even more severe impairment in FADD and caspase 8 recruitment. Finally, in FIG. 15C, a similar correlation between DISC components and receptor function was observed. The complete loss-of-function mutation, L334A, showed a decrease in FADD and caspase 8 (pro- and cleaved) as compared with wild-type; however, the corresponding tumor mutation, L334F, exhibited increased binding of both FADD and caspase 8 in accordance with its classification as a partial loss-of-function mutant. Likewise, the partial loss-of-function mutant, E338A, recruited more FADD and caspase 8 than its complete loss-of-function tumor counterpart, E338K. The correlation between FADD/caspase 8 recruitment to TRAIL DISCs and the cell death data for the complete and partial loss-of-function mutants supports the importance of these residues uncovered in the earlier overexpression studies.

Discussion

Mutagenesis of the death domain of KILLER/DR5 revealed hydrophobic residues expected to be important for overall protein structure as well as charged residues which are expected to interact with downstream effector molecules. The hydrophobic residues tested (Trp-325, Leu-334, Ile-339, Trp-360, and Leu-377) cause a partial (Leu-377) or complete loss-of-function when mutated to alanine. Additional studies suggested that disruption of the Leu-334 Fas complementary residue (lpr) caused a disruption of the protein structure. Future crystal structure studies may determine whether these five hydrophobic residues are truly buried within the protein as expected. Charged residues implicated in mediating electrostatic interactions include Arg-330 (complete loss), Lys-331, Asp-336, Glu-338, Lys-340, Lys-343, and Asp-351. These residues (excluding Glu-338 and Asp-351) were also known to be important for Fas or FADD self-aggregation as well as protein partner binding, signifying their importance in death domain signaling in general. It is possible that mutagenesis of charged residues led only to partial loss-of-function because multiple residual charges were still able to stabilize protein-protein interactions. Careful attention to charge distribution within helices 2 and 3 of the receptors begins to explain differences in receptor/adapter specificity. Residue Glu-326 was known to cause a complete loss in Fas/FADD interaction, yet no apparent defect was observed when mutated to alanine in KILLER/DR5. It is interesting to note the difference in this case because the charge of this particular position varies within the superfamily of receptors. Only the TRAIL receptors, DR4 and KILLER/DR5, have a negatively charged amino acid at this position which illustrates differences between TRAIL and other TNFR family receptors. Position 336, also shown to be important in this study, retains a negative charge in all family members except DR4. Lastly, position 338 has a partial defect in KILLER/DR5-mediated apoptosis.

In order to more specifically examine adapter binding to loss-of-function KILLER/DR5 mutants, this study utilized a TRAIL DISC immunoprecipitation strategy in which various mutants were introduced and the relative levels of FADD and caspase 8 were assessed. The DD mutants were actively recruited into TRAIL DISCs after TRAIL treatment, signifying that cytoplasmic DD loss-of-function mutations had no effect on ligand binding. The defects, however, could be explained by a decrease in both FADD and caspase 8 recruitment into TRAIL DISCs. As expected, the amount of caspase 8 and FADD recruitment directly correlated with the amount of apoptosis induction, as assessed by the cell death assays. These experiments illustrate the concept that one mutant TRAIL receptor (i.e. KILLER/DR5) can potentially disrupt cell death signaling through the formation of defective TRAIL DISCs.

The identification of DD tumor point mutants allowed us to functionally characterize their apoptotic capability and FADD/caspase 8 recruitment in addition to comparing their phenotype to the corresponding alanine substitution. The most prevalent mutation occurred in 3 out of 11 samples (L334F) corresponding to the naturally occurring Fas lpr mutation, demonstrating the conserved importance of this residue in two receptor systems. The alanine substitution yielded a mutant receptor with a greater reduction in apoptotic potential as well as FADD and caspase 8 recruitment as compared with the tumor-derived mutant (L334F). This divergence in phenotype is likely explained by the maintenance of a hydrophobic residue in the tumor-specific mutation. Nevertheless, its prevalence in non-small cell lung cancer points to its importance in maintaining proper protein folding and death signaling. In contrast, the drastic charge change of the tumor mutant E338K resulted in a complete loss-of-death induction by the receptor, whereas the alanine mutant only partially disrupted cell death signaling. The severity of the tumor-specific defect E338K points to an overall disruption of the electrostatic interactions maintained by the residues in helices 2 and 3.

When the loss-of-function L334F, E338K, S324F, E326K, K386N, D407Y mutants were generated and tested, no henotype was observed, supporting the observations made from the alanine mutagenesis. Physiological levels of these mutant receptors are expected to affect their stability or in the context of ligand binding demonstrate defective signaling, but these overexpression studies coupled with the lack of conservation of these residues among family members argue against their importance in KILLER/DR5 structure/function.

In conclusion, the mutational study of the death domain of KILLER/DR5 identified conserved hydrophobic residues and charged amino acids, which are crucial for normal cell death signaling downstream of the receptor. The clustering of specific charged residues critical for apoptosis that are proposed to be in helices 2 and 3 correlates with data from other death domain-containing proteins (Fas, FADD) that these helices are critical for protein/protein interactions. These mutants are expected to be useful in order to better understand the molecular mechanisms behind receptor/adapter specificity.

Detailed Description of FIGS. 10-15

FIG. 10. Alignment of DD from TNF receptor superfamily members. Numbers denote residues in KILLER/DR5 that were targeted in alanine scanning mutagenesis. Bold line below Fas DD denotes a-helices determined from Fas crystal structure studies.

FIG. 11. Protein expression of wild-type (W.T.) and alanine mutant KILLER/DR5 constructs. A, 0.5×10⁶ SW480 cells were transiently transfected for 16 hours, harvested, and equivalent amounts of protein were run on a 15% SDS-polyacrylamide gel. The gel was then transferred and probed with an anti-Myc antibody, followed by a horseradish peroxidase-conjugated secondary and ECL. B, comigration of in vivo upper KILLER/DR5 band with unprocessed in vitro translation (IVT) product. The unlabeled in vitro translation reaction was carried out on 1 ug of plasmid. The in vitro translation reactions were run out along with transfected lysates and processed as in A.

FIG. 12. Example of flow cytometry/GFP-spectrin based assay to assess sub-G₁ peak following transient transfection of wild type (W.T.) and alanine mutant KILLER/DR5 constructs. SW480 cells were transfected with a 1:10 ratio of GFP-spectrin:mutant plasmid and harvested at 18 hours for flow cytometric analysis. Specifically transfected GFP-positive cells are shown in the left column, and untransfected cells are shown in the right column. Sub-G₁ peak, indicative of apoptosis, is denoted by the percentage shown to the left of the vertical line in each graph. One example is given for vector, wild-type KILLER/DR5, signaling competent and incompetent mutant transfected to demonstrate the assay.

FIG. 13. PARP cleavage following transient transfection of wild-type (W.T.) and alanine mutant KILLER/DR5 constructs. Lysates from FIG. 11A were reprobed with an anti-PARP antibody to measure amount of PARP cleavage induced by each mutant. The arrow denotes the cleaved PARP product.

FIG. 14. Protein expression and PARP cleavage induction by tumor-derived KILLER/DR5 mutants. The upper panel displays protein expression of the mutants as assessed by anti-Myc Western blot, and the lower panel measures PARP cleavage induced by each mutant. W.T., wild type.

FIG. 15. A, TRAIL DISC IP of transfected 293 cells. A T75 (80% confluent) of 293 HEK cells was transfected for 16 hours with the indicated wild-type (WT) or mutant KILLER/DR5 plasmid followed by a 15-minute TRAIL treatment (50 ng/ml) at 37° C. The DISC immunoprecipitation was carried out overnight at 4° C. followed by SDS-polyacrylamide gel electrophoresis and Western blot analysis for DISC-associated caspase 8, FADD, and exogenous KILLER/DR5. Total caspase 8 is provided as a loading control to ensure equal amounts of protein were used for TRAIL treatment and DISC analysis. B, TRAIL DISC IP of panel of total and partial loss-of-function alanine mutants. The DISC IPs were carried out as specified in A. The (−) TRAIL lane includes the anti-His6 antibody and protein A/G-agarose beads as a control for the specificity of the DISC IP. C, TRAIL DISC IP of the alanine versus tumor loss-of-function mutants. IPs were carried out as detailed above. P indicates partial loss-of-function, and C indicates complete loss-of-function mutants.

Applicants note that additional experimental details, such as identification of the source/manufacturer of materials and reagents, and primer and probe sequences, are found in U.S. patent application No. 60/232,556 and Applicants' publication, E. Robert McDonald III, Patricia C. Chui, Peter F. Martelli, David T. Dicker, and Wafik S. El-Deiry, Death Domain Mutagenesis of KILLER/DR5 Reveals Residues Critical for Apoptotic Signaling, Journal of Biological Chemistry, 276(18):14939-14945 (2001), the contents of which are incorporated by reference herein in full.

All publications and patent documents referred to herein are incorporated by reference in full, to the same extent as if each individual document was specifically and individually indicated to be incorporated by reference.

The invention being thus described, it will be obvious that the same may be modified or varied in many ways. Such modifications and variations are not to be regarded as a departure from the spirit and scope of the invention and all such modifications and variations are intended to be included within the scope of the following claims.

We claim:

1. A method for modulating cellular apoptosis in an animal by regulating the quantity of functional KILLER/DR5 receptor protein molecules in the target cell(s) of said animal.

2. The method of claim 1, which further comprises: administering to said animal a recombinant viral vector comprising a nucleic acid sequence encoding a wild-type KILLER/DR5 receptor protein or a nucleic acid sequence encoding a loss-of-function mutant KILLER/DR5 receptor protein, said sequence operatively linked to regulatory sequence(s) directing expression of said receptor protein in the target cell(s) of said animal.

3. The method of claim 1, which further comprises: administering to said animal a wild-type KILLER/DR5 receptor protein or a loss-of-function mutant KILLER/DR5 receptor protein.

4. The method of claim 1, wherein the regulation of said protein increases the difference in toxicity response between said target cell(s) and non-target cell(s) in response to a therapy or treatment.

5. The method of claim 4, wherein said target cell(s) are cancer cell(s).

6. The method of claim 5, wherein said cancer cell(s) are spleen cancer cell(s) or gastrointestinal cancer cell(s).

7. The method of claim 4, wherein the regulation of said protein produces an increase in functional KILLER/DR5 receptor protein molecules per target cell.

8. The method of claim 7, wherein said increase in functional KILLER/DR5 receptor protein molecules per target cell is induced by introducing one or more copies of an isolated exogenous wild-type KILLER/DR5 receptor nucleic acid sequence capable of being expressed in said target cell(s).

9. The method of claim 4, wherein the regulation of said protein produces a decrease in functional KILLER/DR5 protein molecules per target cell.

10. The method of claim 9, wherein said decrease in functional KILLER/DR5 receptor protein molecules per target cell is induced by introducing one or more copies of an isolated exogenous mutated KILLER/DR5 receptor nucleic acid sequence which produces loss-of-function KILLER/DR5 mutant receptor proteins in the target cell(s).

11. The method of claim 10, wherein said isolated exogenous mutated KILLER/DR5 receptor nucleic acid sequence is selected from the group consisting of a wild-type KILLER/DR5 receptor nucleic acid sequence bearing one or more point mutations at amino acid(s) 325, 331, 334, 336, 338, 339, 340, 343, 351, and/or 360.

12. The method of claim 11, wherein said target cell(s) is/are selected from the group consisting of spleen cell(s) and gastrointestinal cell(s).

13. The method of claim 1, which additionally comprises a second method for inducing cell death.

14. The method of claim 12, wherein said second method for inducing cell death is a cancer treatment selected from the group consisting of surgical intervention, radiotherapy, hormonal therapy, immunotherapy, chemotherapy, cryotherapy, antineoplastic therapy, and gene therapy.

15. The method of claim 14, wherein said cancer treatment is selected from the group consisting of radiotherapy and chemotherapy.

16. A method for treating a disease or condition in an animal, which comprises:
(a) introducing one or more copies of an isolated exogenous wild-type KILLER/DR5 receptor nucleic acid sequence capable of being expressed, into target cell(s) of said animal; and
(b) treating said animal by administering a therapy selected from the group consisting of radiotherapy and chemotherapy.

17. The method of claim 16, wherein said disease is cancer.

18. The method of claim 16, wherein said nucleic acid sequence and said therapy are administered simultaneously or sequentially.

19. A method for treating a disease or condition in an animal, which comprises:
(a) introducing one or more copies of an isolated exogenous loss-of-function KILLER/DR5 receptor nucleic acid sequence capable of being expressed, into a target spleen cell or small intestine cell of said animal; and
(b) treatment of said animal with a therapy selected from the group consisting of radiotherapy and chemotherapy.

20. The method of claim 19, wherein said disease is cancer.

21. The method of claim 19, wherein said nucleic acid sequence and said therapy are administered simultaneously or sequentially.

22. An isolated nucleic acid sequence for the KILLER/DR5 receptor protein, selected from the group consisting of a wild-type KILLER/DR5 receptor nucleic acid sequence bearing one or more point mutations at amino acid(s) 325, 331, 334, 336, 338, 339, 340, 343, 351, and/or 360.

23. A pharmaceutical composition comprising:
(a) a therapeutically acceptable amount of an isolated nucleic acid sequence for the KILLER/DR5 receptor protein, selected from the group consisting of a wild-type KILLER/DR5 receptor nucleic acid sequence bearing one or more point mutations at amino acid(s) 325, 331, 334, 336, 338, 339, 340, 343, 351, and/or 360; and
(b) a pharmaceutically acceptable carrier.
